(19) United States
(12) Patent Application Publication
(10) Pub. No.: US 2026/0096746 A1
(43) Pub. Date: Apr. 9, 2026

(54) Physiological Monitoring Using Low Sample Rate Accelerometer Data

(71) Applicant: iRhythm Technologies, Inc., San Francisco, CA (US)

(72) Inventors: Yuriko Tamura, San Mateo, CA (US); Elaine Yuiyi Yu, Walnut Creek, CA (US); Jasmine Yu Hu, Shoreline, WA (US); Andrew David Gilbert, Vancouver (CA)

(73) Assignee: iRhythm Technologies, Inc., San Francisco, CA (US)

(21) Appl. No.: 19/347,602

(22) Filed: Oct. 1, 2025

Related U.S. Application Data
(60) Provisional application No. 63/703,833, filed on Oct. 4, 2024.
(60) Provisional application No. 63/703,710, filed on Oct. 4, 2024.

(51) Int. Cl. A61B 5/11; A61B 5/00; A61B 5/0205; A61B 5/107; A61B 5/318; G01P 15/18

(52) U.S. Cl. CPC A61B 5/1126; A61B 5/0205; A61B 5/1071; A61B 5/1118; A61B 5/318; A61B 5/6801; A61B 5/7267; A61B 5/742; G01P 15/18; A61B 2562/0219

(57) ABSTRACT

Techniques for physiological monitoring using low sample rate accelerometer data are described and are implementable to generate insights related to user states during extended wear periods. In an example, low sample rate accelerometer data having a sample rate that is below a sample rate threshold is received from a wearable device mounted on a skin surface of a chest region of a user during a wear period. The low sample rate accelerometer data is processed to extract one or more motion-derived parameters that characterize temporal movement patterns of the user during the wear period. An insight for presentation related to a user state during the wear period is generated based on the one or more motion-derived parameters. The insights can include but are not limited to predictions of sleep states, active states, and inactive states, body angle and position determinations, and detection of device inversion events.

### RELATED APPLICATIONS

This application claims priority to U.S. Provisional Application No. 63/703,833, filed Oct. 4, 2024, and titled “Sleep and Activity Prediction with Constrained Conditions,” and to U.S. Provisional Application No. 63/703,710, filed Oct. 4, 2024, and titled “Body and Device Angle and Position Detection,” which are hereby incorporated by reference in their entireties.

### BACKGROUND

Wearable devices for monitoring physiological parameters and activity patterns have become increasingly prevalent in healthcare and wellness applications and enable healthcare providers and individuals to track health metrics without frequent clinical visits or disruption to daily activities. These devices typically employ various sensors and monitoring components to track physiological parameters, behavioral patterns, and/or activity levels over extended periods. However, wearable monitoring devices may face challenges that limit effectiveness for continuous, long-term monitoring applications. For instance, computational overhead involved in real-time collection, processing, and analysis of sensor data can cause power consumption challenges, which may reduce battery life and shorten monitoring duration and thus offset the advantages provided by wearable devices.

### BRIEF DESCRIPTION OF THE DRAWINGS

FIG. 1 is a block diagram of a non-limiting example of an environment that is operable to employ systems as described herein.

FIG. 2 depicts a non-limiting example of a monitoring device.

FIG. 3 depicts a non-limiting system in an example implementation of physiological monitoring using low sample rate accelerometer data showing operation of the prediction system of FIG. 1 in more detail.

FIG. 4 depicts a nonlimiting example of physiological monitoring using low sample rate accelerometer data in which insights are generated that include a sleep/activity state.

FIG. 5 depicts a nonlimiting example of physiological monitoring using low sample rate accelerometer data in which body angles and positions are generated based on accelerometer data.

FIGS. 6a, 6b, and 6c depict nonlimiting examples of physiological monitoring using low sample rate accelerometer data in which a machine learning system is trained, configured, and implemented to generate insights based on various parameters.

FIG. 7 depicts a nonlimiting example of physiological monitoring using low sample rate accelerometer data in which device inversion can cause measurement inaccuracies.

FIG. 8 depicts a nonlimiting example of physiological monitoring using low sample rate accelerometer data including detection of device inversion events.

FIGS. 9a-9e depict non-limiting examples of physiological monitoring using low sample rate accelerometer data in which in which a report that includes various insights is output in health report interfaces.

FIG. 10 depicts a flow diagram depicting an algorithm as a step-by-step procedure in an example implementation that is performable by a processing device to generate insights related to conditions of wear periods based on low sample rate accelerometer data from a wearable device.

FIG. 11 depicts a flow diagram depicting an algorithm as a step-by-step procedure in an example implementation that is performable by a processing device to generate insights related to user states based on low sample rate accelerometer data.

FIG. 12 depicts a flow diagram depicting an algorithm as a step-by-step procedure in an example implementation that is performable by a processing device to determine body angle and position based on low sample rate accelerometer data collected during extended wear periods.

FIG. 13 depicts a flow diagram depicting an algorithm as a step-by-step procedure in an example implementation that is performable by a processing device to integrate ECG and accelerometer data for enhanced sleep and activity prediction using machine learning.

FIG. 14 depicts a flow diagram depicting an algorithm as a step-by-step procedure in an example implementation that is performable by a processing device to detect device inversion events during extended monitoring periods.

### DETAILED DESCRIPTION

Conventional activity tracking devices often include accelerometers that operate at relatively high sampling frequencies, typically ranging from 25 to 100 Hertz (“Hz”), to capture motion data for activity analysis. However, such high-frequency sampling creates substantial power consumption demands that limit battery life and reduce monitoring duration for continuous wear applications. For instance, power limitations may result in frequent charging requirements that interrupt monitoring continuity, limit an amount of analyzable data, and reduce user compliance, particularly in clinical applications where extended wear periods are needed for accurate health assessment. Further, factors such as individual variations in movement patterns, device placement, and signal noise can impact accuracy of such devices.

Accordingly, techniques, methods, and systems for physiological monitoring using low sample rate accelerometer data are described that overcome these limitations by enabling accurate generation of various insights such as user states and device properties while operating under constrained power and memory conditions. By way of example, a prediction system leverages a wearable device positioned on a chest region of a user that collects accelerometer data at a relatively low sampling rate (e.g., approximately 1.56 Hz) over an extended wear period, e.g., from one to fourteen days. Because the sampling rate is significantly reduced relative to conventional techniques, the wearable device is able to collect measurements for a duration of the wear period without device recharging or battery replacement.

In various examples, the low sample rate accelerometer data is processed to extract motion-derived parameters that characterize temporal movement patterns of the user. The motion derived parameters, for instance, include calculated values extracted from the raw accelerometer data that quantify aspects of user movement and position over time. The motion derived parameters can include metrics such as acceleration magnitude, standard deviations of the acceleration magnitude over various temporal intervals, reference and/or position vectors that correspond to a body angle, rolling averages of one or more components of the accelerometer data, and so forth.

Based on the motion-derived parameters, the prediction system can generate various insights related to conditions of the wear period and/or user states during the wear period. For instance, the prediction system is operable to generate insights that include predictions of sleep states, active states, and inactive states at discrete (e.g., minute-level) resolution throughout the wear period. Thus, the system can determine “when” a user is asleep, engaged in activity, or is awake but inactive throughout the wear period.

The system is further able to determine a body angle and/or body position of the user based on the low sample rate accelerometer data. In an example to do so, the system computes a reference vector from a high activity period when the user is likely upright and position vectors that correspond to user positions throughout the wear period. The system calculates body angles as polar angles in a spherical coordinate system between the reference vector and the position vectors, where zero degrees represents upright positioning and ninety degrees represents recumbent positioning.

The system can further determine body positions of the user by classifying calculated body angles into positional categories using predefined angle thresholds. For instance, body angles near zero degrees may indicate an upright or standing position, intermediate angles (e.g., between 30 and 60 degrees) may correspond to a reclined position, and angles approaching ninety degrees may represent lying down or supine positions. In some implementations, the system leverages the determined body angle and position to inform sleep and activity predictions. For example, the system can determine that a recumbent position during a period of relatively low movement indicates an inactive state, while an upright position with increased motion may correspond to an active state.

In various examples, the system can further determine an orientation of the wearable device, such as to detect device inversion events during the wear period. For instance, the wearable device may become flipped or rotated from an intended orientation during initial application or during mid-wear reattachment or repositioning. Such inversion events may impact accuracy of accelerometer-based predictions and physiological measurements. Accordingly, the system can leverage rolling averages of one or more accelerometer axis components to detect and enable automatic correction of signal polarity such as to maintain data integrity throughout extended monitoring sessions.

Further, the system can leverage electrocardiogram (“ECG”) data collected during the wear period to inform insights and/or to generate correlations between ECG derived insights and accelerometer-based insights. In some examples, the system leverages a machine learning system to do so that includes using separate trained machine learning models/algorithms. The machine learning system can combine accelerometer-based and ECG-based predictions such as through heuristic combination schemes to enhance accuracy. By applying machine learning algorithms that are trained using low sample rate training data and reinforced with ECG derived predictions, the techniques described herein support generation of insights not possible under constrained conditions using conventional approaches that merely down sample high-frequency algorithms.

The system is further operable to configure the generated insights for presentation, such as in comprehensive reports that are output during and/or upon conclusion of the wear period. A report, for instance, can include various information such as summary sections that depict aggregate user state data and/or daily breakdown sections that correlate sleep and activity information with physiological data such as heart rate patterns. These reports enable users and healthcare providers to visualize patterns and trends in patient behavior and physiological responses over extended monitoring periods, which facilitates informed clinical decision-making.

In this way, the techniques, methods, and systems described herein provide significant advantages over conventional systems by achieving enhanced monitoring accuracy while consuming substantially less power. Thus, these techniques enable continuous monitoring for extended wear periods (e.g., 14 days) without recharging, which overcomes limitations of conventional devices that are reliant on daily charging. Further, the techniques described herein support insight generation and/or signal processing adjustments based on one or more of a variety of interconnected factors such as sleep/activity states, body position, device orientation, and ECG measurements to provide clinically relevant and actionable information.

In some aspects, the techniques described herein relate to a method including: receiving low sample rate accelerometer data having a sample rate that is below a sample rate threshold collected by a wearable device attached to a skin surface of a chest region of a user during a wear period; processing the low sample rate accelerometer data to extract one or more motion-derived parameters that characterize temporal movement patterns of the user during the wear period; and generating, based on the one or more motion-derived parameters, an insight for presentation related to a user state during the wear period.

In some aspects, the techniques described herein relate to a method, wherein the low sample rate accelerometer data is collected at a sample rate of approximately 1.56 Hz and the wear period is between one and fourteen days.

In some aspects, the techniques described herein relate to a method, wherein the insight includes predictions of sleep states, active states, and inactive states of the user during one or more temporal intervals of the wear period.

In some aspects, the techniques described herein relate to a method, wherein the one or more motion-derived parameters include an acceleration magnitude for a particular temporal interval of the wear period calculated as a square root of a sum of squares of one or more acceleration components of the low sample rate accelerometer data and an activity parameter calculated as a standard deviation of the acceleration magnitude for the particular temporal interval, wherein a relatively low standard deviation corresponds to user stillness and a relatively high standard deviation corresponds to user movement.

In some aspects, the techniques described herein relate to a method, wherein the insight includes a body angle or body position of the user during one or more temporal intervals of the wear period.

In some aspects, the techniques described herein relate to a method, wherein the one or more motion-derived parameters include a reference vector that corresponds to an upright position of the user generated based on portions of the low sample rate accelerometer data that indicate relatively high activity, and the body angle is calculated as a polar angle in a spherical coordinate system between the reference vector and a position vector for a particular temporal instance of the wear period.

In some aspects, the techniques described herein relate to a method, wherein the insight includes a detection of inversion events of the wearable device during the wear period and is generated based on motion-derived parameters that include rolling averages of accelerometer axis components of the low sample rate accelerometer data over a particular temporal interval of the wear period.

In some aspects, the techniques described herein relate to a method, further including configuring the insight for presentation in a user interface as part of a report that includes: a summary section that depicts aggregate user state data for the wear period; and a daily breakdown section that depicts the user state in temporal correlation with physiological data collected during the wear period.

In some aspects, the techniques described herein relate to a method, further including receiving electrocardiogram (“ECG”) data collected by the wearable device, and wherein generating the insight is further based on the ECG data.

In some aspects, the techniques described herein relate to a processing device including: one or more processors; and memory having stored computer-readable instructions that are executable by the one or more processors to perform operations including: receiving accelerometer data having a sample rate that is below a sample rate threshold, the accelerometer data collected by an accelerometer of a wearable device attached to a skin surface of a user during a wear period; processing the accelerometer data to extract one or more motion-derived parameters that characterize temporal movement patterns of the user during the wear period; and generating, based on the one or more motion-derived parameters, an insight for presentation related to a condition of the wear period.

In some aspects, the techniques described herein relate to a processing device, wherein the accelerometer data is collected at a sample rate of approximately 1.56 Hz and the wear period is between one and fourteen days.

In some aspects, the techniques described herein relate to a processing device, wherein the insight includes sleep states, active states, and inactive states of the user throughout the wear period.

In some aspects, the techniques described herein relate to a processing device, wherein the insight includes a body angle or body position of the user throughout the wear period.

In some aspects, the techniques described herein relate to a processing device, wherein the insight includes a detection of whether an inversion event to the wearable device has occurred during the wear period.

In some aspects, the techniques described herein relate to a processing device, the operations further including receiving electrocardiogram (“ECG”) data collected by an ECG sensor of the wearable device and generating the insight based in part on the ECG data.

In some aspects, the techniques described herein relate to a processing device, the operations further including configuring the insight for presentation in a user interface as part of a report that includes a summary section depicting aggregate user state data for the wear period and a daily breakdown section that includes heart rate data overlaid on sleep and activity data.

In some aspects, the techniques described herein relate to a system including: an accelerometer sensor of a wearable device configured to collect low sample rate accelerometer data via contact with a skin surface of a user during a wear period; and one or more processors configured to: receive the low sample rate accelerometer data, the low sample rate accelerometer data having a sample rate that is below a sample rate threshold; process the low sample rate accelerometer data to extract one or more motion-derived parameters that characterize temporal movement patterns of the user during the wear period; and present an insight generated based on the one or more motion-derived parameters that indicates a user state during the wear period.

In some aspects, the techniques described herein relate to a system, wherein the insight includes one or more of a sleep or activity state of the user, a body position of the user, or a device inversion event of the wearable device during the wear period.

In some aspects, the techniques described herein relate to a system, further including one or more electrocardiogram (ECG) sensors of the wearable device, wherein the one or more processors are configured to receive ECG data collected by the ECG sensor and determine the insight based on the ECG data and the low sample rate accelerometer data.

In some aspects, the techniques described herein relate to a system, wherein the one or more processors are configured to process the low sample rate accelerometer data by applying a trained machine learning algorithm that has been trained on historical accelerometer data and corresponding user state labels to extract the one or more motion-derived parameters and generate the insight.

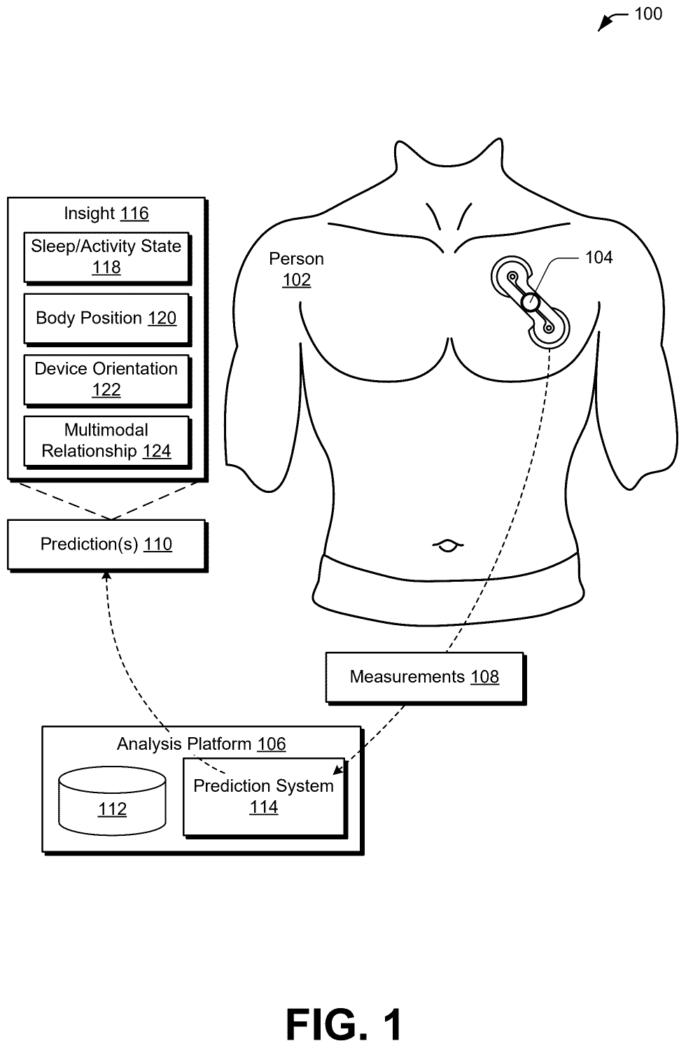

FIG. 1 is a block diagram of a non-limiting example 100 of an environment that is operable to employ systems as described herein. The illustrated example 100 includes person 102, who is depicted wearing a monitoring device 104, i.e., a wearable device. The illustrated environment also includes an analysis platform 106. The analysis platform 106 may be connected to the monitoring device 104 via one or more wireless connections directly or via one or more wired and/or wireless connections and one or more intermediate devices, such as a computing device associated with the person 102, network routing devices and equipment, server devices, and/or the Internet, to name just a few.

The monitoring device 104 may be utilized to monitor one or more aspects of the person 102, such as to generate measurements 108. In some scenarios, for instance, the monitoring device 104 may be provided to record electrical activity of the person 102's heart over an observation period, e.g., lasting some number of seconds or minutes, lasting multiple days, and so on. By way of example, the person 102 may have a magnitude of his or her heart's electrical potential monitored over time to produce one or more electrocardiograms, which may be used to predict any of a variety of events. In at least one example, the monitoring device 104 is provided to record accelerometer and/or electrocardiogram (“ECG”) measurements over an observation period.

Alternatively or in addition, the monitoring device 104 may be used to output measurements 108 (e.g., a time sequence of measurements such as a time sequence of electric potential measurements), which may indicate an observation or be used to generate a prediction of one or more events.

In connection with the monitoring device, instructions may be provided to the person 102 that instruct the person 102 how to operate the monitoring device 104 and/or how to behave (e.g., sleep, perform activity) while wearing monitoring device 104. In one or more implementations, the instructions may be provided as part of a kit, e.g., written instructions. Alternately or additionally, the analysis platform 106 may cause the instructions to be communicated to and output (e.g., for display and/or audio output) via a computing device associated with the person 102. In one or more implementations, the analysis platform 106 may wait to provide these instructions for output after a predetermined amount of time of an observation period has lapsed (e.g., two days) while wearing the monitoring device 104 and/or based on patterns in the aspects of the person 102 being measured.

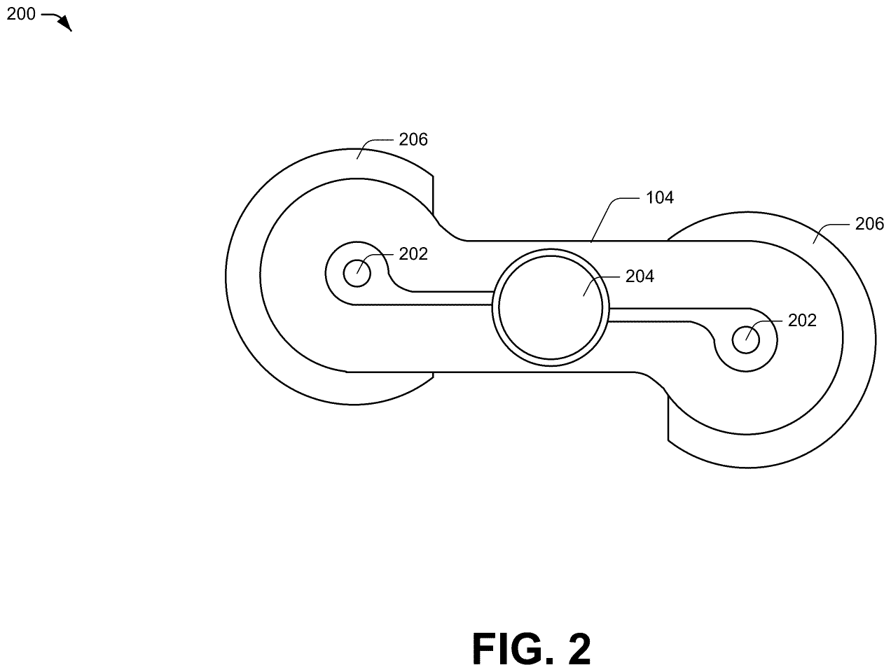

The monitoring device 104 may be configured in a variety of ways to monitor one or more aspects of the person 102. Moreover, the form factor depicted in FIGS. 1 and 2 is just one example form factor, and the form factor of the monitoring device 104 may differ in variations. It is to be appreciated that the monitoring device 104 may be configured with one or more sensors, examples of which include one or more of: a plurality of electrodes (e.g., that can be placed on the skin of the person), an accelerometer, and a pulse oximeter (e.g., to measure and record oxygen saturation (SpO2) and/or produce a photoplethysmogram of the person 102), to name just a few. Certainly, the monitoring device 104 may be configured with any of a variety of types of sensors without departing from the described techniques.

Although the monitoring device 104 may be configured in a similar manner as monitoring devices used for clinically monitoring patients, in one or more implementations, the monitoring device 104 may be configured differently than the devices used for monitoring and/or diagnosing patients clinically. By way of example, and not limitation, the monitoring device 104 may be configured as a ring, a watch, a patch, and/or a strap, to name just a few form factors. Alternatively or additionally, the monitoring device 104 may have a similar form factor as for clinical settings, but have different functionality, such as functionality that prevents a wearer from viewing the measurements 108.

In one or more implementations, the monitoring device 104 may be configured to offload measurements 108 during the course of the observation period. By way of example, the monitoring device 104 may offload the measurements 108 by transmitting them via a wired or wireless connection to an external computing device, e.g., at predetermined time intervals and/or responsive to establishing or reestablishing a connection with the computing device. In one or more implementations, the measurements 108 and/or other data from the monitoring device 104 may be compressed by the monitoring device 104 for wireless transmission, e.g., using one or more of a variety of data compression techniques. Compression of the sensor data in this way can reduce battery usage of the monitoring device 104 during the observation period and facilitate wear during assessments of physiological conditions.

To the extent that the monitoring device 104 may be configured to store the measurements 108 for an entirety of an observation period, in one or more implementations, the monitoring device 104 may be configured without wireless transmission means, e.g., without any antennae to transmit the measurements 108 wirelessly and without hardware or firmware to generate packets for such wireless transmission. Instead, the monitoring device 104 may be configured with hardware to communicate the measurements 108 via a physical, wired coupling. In such scenarios, the monitoring device 104 may be “plugged in” to extract the measurements 108 from the device's storage.

Accordingly, the monitoring device 104 may be configured with one or more ports to enable wired transmission of the measurements 108 to an external computing device. Examples of such physical couplings may include micro universal serial bus (USB) connections, mini-USB connections, and USB-C connections, to name just a few. Although the monitoring device 104 may be configured for extraction of the measurements 108 via wired connections as discussed just above, in different scenarios, the monitoring device 104 may alternately or additionally be configured to offload the measurements 108 over one or more wireless connections.

Once the monitoring device 104 produces the measurements 108, the measurements 108 are provided to the analysis platform 106. As noted above, the measurements 108 may be communicated to the analysis platform 106 over wired and/or wireless connection(s).

In scenarios where the analysis platform 106 is implemented partially or entirely on the monitoring device 104, for instance, the measurements 108 may be transferred over a bus from the device's local storage to a processing system of the device. In scenarios where the monitoring device 104 is configured to generate one or more predictions 110 by processing the measurements 108, the monitoring device 104 may also be configured to provide the generated one or more predictions 110 as output, e.g., by communicating the one or more predictions 110 to an external computing device. In other scenarios, the measurements 108 may be processed by an external computing device configured generate one or more predictions 110. For example, the measurements 108 may be processed by a smartphone associated with the user, a smartphone or other dedicated device associated with the monitoring device 104, and/or one or more server computers at a data center or other location that can be utilized by an entity associated with the monitoring device 104, to name just a few. In other words, those other devices may implement at least a portion of the analysis platform 106 and/or a prediction system 114.

In one or more implementations, the monitoring device 104 is configured to transmit the measurements 108 to an external device over a wired connection with the external device, e.g., via USB-C or some other physical, communicative coupling. Here, a connector may be plugged into the monitoring device 104 or the monitoring device 104 may be inserted into an apparatus having a receptacle that interfaces with corresponding contacts of the device. The measurements 108 may then be obtained from storage of the monitoring device 104 via this wired connection, e.g., transferred over the wired connection to the external device. Such a connection may be used in scenarios where the monitoring device 104 is mailed by the person 102 after the observation period, such as to a health care provider, telemedicine service, provider of the monitoring device 104, or medical testing laboratory.

Alternatively or additionally, the monitoring device 104 may provide the measurements 108 to the analysis platform 106 by communicating the measurements 108 over one or more wireless connections. For example, the monitoring device 104 may wirelessly communicate the measurements 108 to external computing devices, such as a mobile phone, tablet device, laptop, smart watch, other wearable health tracker, and so on. Accordingly, the monitoring device 104 may be configured to communicate with external devices using one or more wireless communication protocols or techniques. By way of example, the monitoring device 104 may communicate with external devices using one or more of Bluetooth (e.g., Bluetooth Low Energy links), near-field communication (NFC), Long Term Evolution (LTE) standards such as 5G, and so forth. Monitoring devices 104 may be configured with corresponding antennae and other wireless transmission means in scenarios where the measurements 108 are communicated to an external device for processing. In those scenarios, the measurements 108 may be communicated to the analysis platform 106 in various manners, such as at predetermined time intervals (e.g., every day, every hour, or every five minutes), responsive to occurrence of some event (e.g., filling a storage buffer of the monitoring device 104), or responsive to an end of an observation period, to name just a few.

Thus, regardless of where the analysis platform 106 is implemented (e.g., at the monitoring device 104, at a smartphone associated with the person 102, or at a server device), the analysis platform 106 obtains the measurements 108 produced by the monitoring device 104. In one or more implementations, the analysis platform 106 also obtains other measurements produced by the monitoring device 104 and/or any other devices used during the observation period, e.g., a smartwatch, chest strap, etc.

In one or more implementations, the analysis platform 106 may be implemented in whole or in part at the monitoring device 104. Alternately or additionally, the analysis platform 106 may be implemented in whole or in part using one or more computing devices external to the monitoring device 104, such as one or more computing devices associated with the person 102 (e.g., a mobile phone, tablet device, laptop, desktop, or smart watch) or one or more computing devices associated with a service provider (e.g., a health care provider, a telemedicine service, a service corresponding to the provider of the monitoring device 104, a medical testing laboratory service, and so forth). In the latter scenario, the analysis platform 106 may be implemented at least in part on one or more server devices.

In the illustrated example 100, the analysis platform includes storage device 112. In accordance with the described techniques, the storage device 112 is configured to maintain the measurements 108 and/or other measurements or information processed by the prediction system 114 to generate one or more predictions 110. The storage device 112 may represent one or more databases and also other types of storage capable of storing the measurements 108 and/or other types of measurements. The storage device 112 may also store a variety of other data, such as personal information, demographic information describing the person 102, information about a health care provider, information about an insurance provider, payment information, prescription information, determined health indicators, account information (e.g., username and password), and so forth. The storage device 112 may also maintain data of other users of a user population, and/or data to support operation of one or more machine learning systems.

In the illustrated example 100, the analysis platform 106 also includes the prediction system 114. The prediction system 114 represents functionality to process the measurements 108 to generate the one or more prediction(s) 110. Alternatively or in addition, the prediction system 114 may output one or more time sequences indicating an observation or prediction of one or more events, over time. It is also to be appreciated that the prediction system 114 may output different combinations of multiple predictions in variations.

In at least one implementation, the prediction system 114 uses machine learning to generate one or more predictions 110. By way of example and not limitation, the prediction system 114 may include one or more neural networks trained based on the historical measurements and the historical outcome data of a user population. The prediction system 114 may include one or multiple machine learning models (e.g., an ensemble of models and/or algorithms). Alternatively or additionally, the prediction system 114 may include logic (a machine learning model and/or other types of logic) to pre-process the obtained measurements, such as to extract various cardiovascular and/or other features from the sequences of measurements. The illustrated example 100 also includes prediction(s) 110, which corresponds to the output of the prediction system 114.

In various examples, the prediction 110 includes and/or is representative of an insight 116. The insight 116 may represent a determination, classification, and/or analysis derived from the measurements 108 that provides information about a condition of the wear period, such as a state and/or characteristic of the person 102 and/or the monitoring device 104 during the observation period. By way of example and not limitation, the insight 116 can include one or more of a sleep/activity state 118, a body position 120, a device orientation 122, and/or a multimodal relationship 124.

As further described in more detail below, the sleep/activity state 118 can represent a classification of a behavioral or physiological state of the person 102 during the observation period, such as whether the person 102 is asleep, actively engaged in physical movement, or awake but inactive. The body position 120 can represent a determination of a physical orientation or posture of the person 102 during the observation period, such as whether the person 102 is upright, reclined, or lying down. The device orientation 122 can represent a determination of a physical position or alignment of the monitoring device 104 during the observation period, such as whether the device has been inverted or rotated from its intended orientation. The multimodal relationship 124, for instance, can represent a correlation or integration between multiple data sources such as accelerometer data and ECG data, enabling enhanced prediction accuracy through combined analysis of different physiological and motion parameters.

In various examples, one or more operations of the analysis platform 106 and/or the prediction system 114 are performable by the monitoring device 104 and/or by one or more devices not physically connected to the monitoring device 104 in substantially real time and/or as post processing operations.

FIG. 2 depicts a non-limiting example 200 of a monitoring device. The illustrated example 200 depicts the monitoring device 104.

In accordance with the described techniques, the monitoring device 104 includes one or more sensors 202, examples of which include but are not limited to one or more pairs of electrodes, an accelerometer, a pulse oximeter, and sweat sensors, to name just a few. The monitoring device 104 may also include a transmitter 204. In this example 200, the monitoring device 104 further includes one or more adhesive portions 206. In operation, the monitoring device 104 is configured to be applied to the skin via the one or more adhesive portions 206, such that, for example, the one or more sensors 202 are positioned to detect and record the electrical activity of the person 102's heart, e.g., to produce an electrocardiogram (ECG and/or EKG). In at least one implementation, the monitoring device 104 may be removed by peeling the one or more adhesive portions 206 off of the skin.

It is to be appreciated that the monitoring device 104 and its various components are simply one form factor, and the monitoring device 104 and its components may have different form factors without departing from the spirit or scope of the described techniques.

In one or more implementations, the monitoring device 104 may include a processor and/or memory (not shown). The monitoring device 104, by leveraging the processor, may generate the measurements 108 based on the communications with one or more sensors 202 that are indicative of some aspect of the person 102, such as the person 102's heart's physical activity, sleep state, electrical activity, etc. In one or more implementations, the processor further generates one or more communicable packages of data that include one or more of the measurements 108 and/or other measurements, such as low sample rate accelerometer data and ECG measurements. Alternately or additionally, the processor produces and/or causes storage of other data, which may be used for predicting classifications of physiological conditions, e.g., sleep apnea.

In implementations where the monitoring device 104 is configured for wireless transmission, the transmitter 204 may transmit the measurements 108 wirelessly as a stream of data to a computing device. In one or more implementations, for instance, the monitoring device 104 is configured to transfer (e.g., transmit and/or receive) information (e.g., electrical potential measurements) via a Bluetooth Low Energy (BLE) connection. Alternately or additionally, the monitoring device 104 may buffer the measurements 108 (e.g., in memory) and cause the transmitter 204 to transmit the buffered measurements later at various intervals, e.g., time intervals (every second, every thirty seconds, every minute, every five minutes, every hour, and so on), storage intervals (when the buffered measurements reach a threshold amount of data), and so forth.

### Physiological Monitoring Using Low Sample Rate Accelerometer Data

The following discussion describes techniques that are implementable utilizing the previously described systems and devices. Aspects of each of the procedures can be implemented in hardware, firmware, software, or a combination thereof. The procedures are shown as a set of blocks that specify operations that can be performed by one or more devices and are not necessarily limited to the orders shown for performing the operations by the respective blocks. One or more blocks of the procedures, for instance, specify operations that can be programmable by hardware (e.g., processor, microprocessor, controller, firmware) as instructions thereby creating a special purpose machine for carrying out an algorithm as illustrated by the flow diagram. As a result, the instructions are storable on a computer-readable storage medium that causes the hardware to perform the algorithm. In portions of the following discussion, reference will be made to FIGS. 1-14.

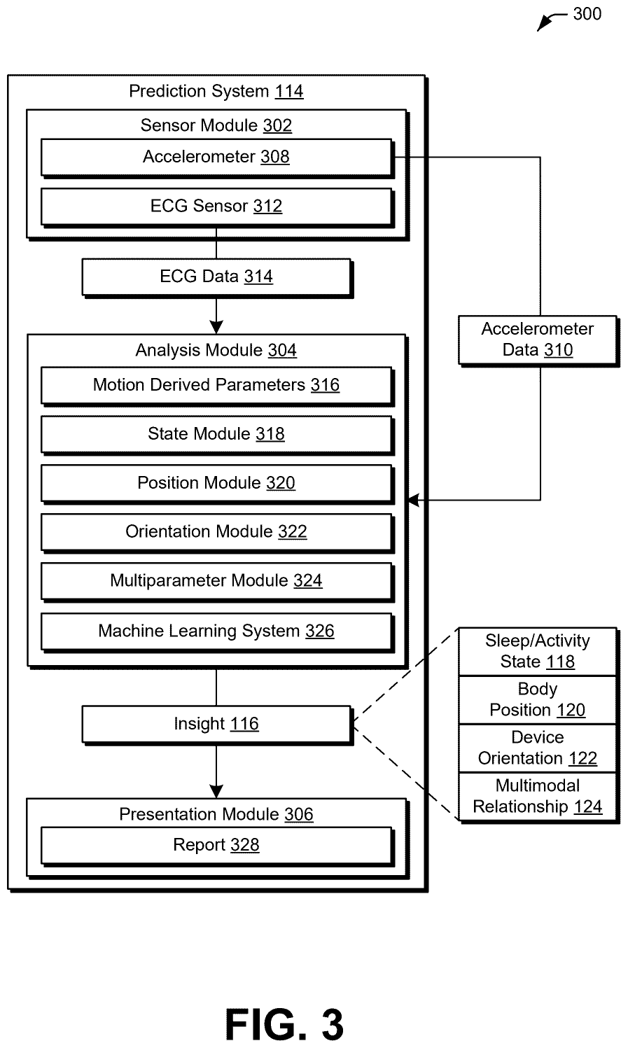

FIG. 3 depicts a non-limiting system in an example implementation 300 of physiological monitoring using low sample rate accelerometer data showing operation of the prediction system 114 of FIG. 1 in more detail. In various examples, the prediction system 114 is representative of, supports functionality of, is implementable by, and/or includes (either partially or wholly) a wearable device, such as the monitoring device 104.

The prediction system 114, for instance, represents a system architecture for processing low sample rate accelerometer data and generating various insights 116 related to a condition of a wear period, such as one or more user states while a monitoring device 104 is attached to a skin surface of a user. The prediction system 114 is depicted to include multiple interconnected modules such as a sensor module 302, an analysis module 304, and a presentation module 306 that are configurable to collect, process, present, and derive insights from physiological data collected from a user during an extended wear period, e.g., one to fourteen days. In various examples, the prediction system 114 may record data for the extended wear period on a single battery without recharging, and thus the techniques described herein enable continuous monitoring while maintaining power efficiency through low sample rate data collection and processing techniques.

For example, the sensor module 302 houses one or more data collection components for acquiring physiological measurements from the user. As illustrated, the sensor module 302 includes an accelerometer 308 that is configured to collect accelerometer data 310 during the wear period. The accelerometer 308 may be implemented as one or more of a variety of motion sensing devices, such as a microelectromechanical systems (“MEMS”) accelerometer, a piezoelectric accelerometer, or a capacitive accelerometer, and may be configured to detect acceleration forces along one or more axes. In some examples, the accelerometer 308 may be a three-axis accelerometer that includes x, y, and z components, however single-axis, dual-axis, or various multi-axes configurations are also contemplated.

The accelerometer data 310, for instance, is low-rate accelerometer data that is below a sample rate threshold. By way of example and not limitation, the sample rate threshold may be approximately 5 Hz, 3 Hz, 2 Hz, or 1.56 Hz, however other sample rates are contemplated without departing from the scope of the described techniques. In various embodiments, the prediction system 114 maintains the sample rate threshold between a particular range, e.g., between 1.0 Hz and 2.0 Hz. In at least one example, the accelerometer data 310 is sampled at variable rates throughout the wear period, such as due to one or more detected conditions. The relatively low sample rate enables extended monitoring periods, while maintaining sufficient data resolution to support generation of insights 116 in accordance with the data processing techniques described herein.

The sensor module 302 is further depicted to include an ECG sensor 312. The ECG sensor 312, for instance, includes one or more electrodes and/or processing elements configured to collect electrocardiogram (“ECG”) measurements and produce ECG data 314. The ECG sensor 312 may be implemented using various electrode configurations (e.g., a two-electrode system, three-electrode system, additional multi-electrode arrangements, etc.) that can be positioned on a chest region of the user to detect cardiac electrical activity. In some implementations, the ECG sensor 312 may include signal conditioning circuitry, amplifiers, and analog-to-digital converters to process the raw electrical signals from the heart into digital ECG data 314.

The ECG data 314 may capture various cardiac parameters including heart rate, rhythm patterns, and waveform characteristics that can be analyzed in conjunction with the accelerometer data 310 to provide multimodal physiological insights. The ECG data 314 may be sampled at various sampling rates and may be processed using a variety of filtering and/or noise reduction techniques, such as to improve signal quality during extended wear periods. As described in more detail below, the prediction system 114 is operable to generate various multi-modal insights based on the accelerometer data 310 and the ECG data 314.

For instance, the analysis module 304 is operable to receive one or more of the accelerometer data 310 and/or the ECG data 314 and performs various data processing operations to generate an insight 116. The insight 116, for instance, may represent a determination, classification, and/or analysis derived from the measurements 108 that provides information about a condition of the wear period, such as a state and/or characteristic of the person 102 and/or the monitoring device 104 during the observation period. By way of example and not limitation, the insight 116 can include one or more of a sleep/activity state 118, a body position 120, a device orientation 122, and/or a multimodal relationship 124.

To do so, the analysis module 304 processes the low sample rate accelerometer data 310 to extract one or more motion-derived parameters 316 that characterize temporal movement patterns of the user during the wear period. The motion-derived parameters 316, for instance, represent calculated values extracted from the raw accelerometer data 310 that quantify aspects of user movement and position over time. The analysis module 304 can leverage one or more of a state module 318, position module 320, orientation module 322, multiparameter module 324, and/or a machine learning system 326 to generate the insight 116 based on various motion derived parameters 316. Accordingly, the particular motion-derived parameters 316 that are generated may vary based on the particular insights 116 being generated, such that different parameter sets may be computed depending on whether the prediction system 114 is to generate sleep/activity states 118, body positions 120, device orientations 122, multimodal relationships 124, or combinations thereof.

For instance, the state module 318 is operable to analyze motion-derived parameters 316 to determine sleep/activity states 118, e.g., sleep states, active states, and/or inactive states of the user throughout the wear period. In an example, the state module 318 generates and/or processes motion-derived parameters 316 such as acceleration magnitude and activity parameters to classify user states. For instance, the state module 318 may calculate a total acceleration magnitude as a square root of a sum of squares of acceleration components in three orthogonal axes of the accelerometer data 310. The state module 318 may further generate an activity parameter as a standard deviation of the acceleration magnitude over predetermined time windows. In some examples, relatively low standard deviation values may indicate user stillness corresponding to inactive or sleep states, while relatively high standard deviation values may indicate movement corresponding to active states.

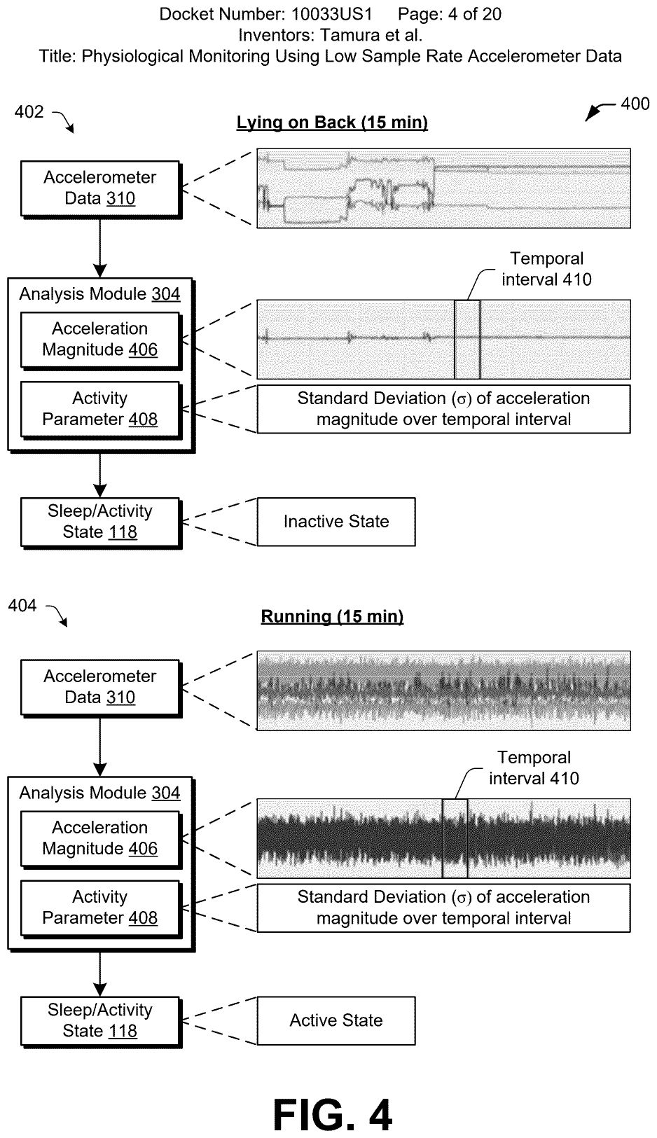

The state module 318 may further distinguish between active and inactive awake states by applying threshold values to the motion-derived parameters 316, such as to enable minute-level resolution of sleep/activity state 118 classifications throughout the extended wear period. In some examples, the state module 318 may incorporate additional insights 116 from other modules within the analysis module 304, such as body angle information from the position module 320, to enhance accuracy of sleep/activity state 118 determinations by providing contextual information about user posture during different activity periods. Functionality of the state module 318 is further discussed in more detail below with respect to FIG. 4.

The position module 320 is operable to analyze motion-derived parameters 316 to determine body angles and/or body positions 120 of the user throughout the wear period. In an example, the position module 320 generates and/or processes motion-derived parameters 316 that include reference vectors and/or position vectors to calculate body positioning information. For instance, the position module 320 may compute a reference vector from high activity periods when the user is likely upright and position vectors that correspond to user positions at various instances throughout the wear period. The position module 320 may further calculate body angles as polar angles in a spherical coordinate system between the reference vector and a particular position vector. The polar angle may range between 0 degrees (e.g., with a position vector parallel to the reference vector) and 90 degrees, e.g., with the position vector substantially perpendicular to the reference vector.

The position module 320 may further determine body positions 120 of the user by classifying calculated body angles into positional categories using predefined angle thresholds. For instance, body angles between 0 and 15 degrees may correspond to an upright or standing position, intermediate angles greater than 15 degrees and less than 85 degrees may correspond to a reclined position, and angles between 85 and 90 degrees may represent lying down or supine positions. This is by way of example and not limitation, and various angle threshold ranges are considered.

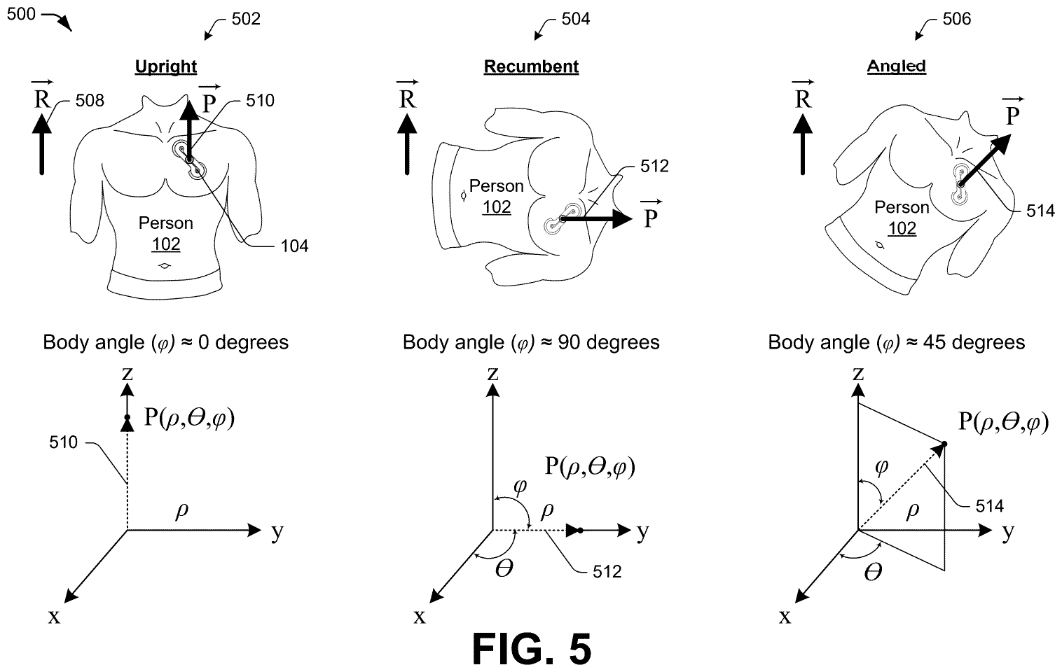

In some implementations, the position module 320 leverages the determined body angle and body position 120 to inform sleep and activity predictions generated by other modules within the analysis module 304. For example, the position module 320 can determine that a recumbent position during a period of relatively low movement indicates a sleep state, an upright position with relatively low movement indicates an awake but inactive state, while an upright position with increased motion may correspond to an active state. The position module 320 is operable to process the accelerometer data 310 to calculate body angles and detect body positions 120 of the user during various temporal intervals of the wear period. Example functionality of the position module 320 is discussed below with respect to FIG. 5.

The orientation module 322 is operable to analyze motion-derived parameters 316 to determine device orientation 122 and detect inversion events of the wearable device during the wear period. An inversion event, for instance, refers to a situation in which the wearable device is attached to the skin surface in an “upside down” position, such as during initial application of the device and/or during the wear period following detachment of the device. In an example, the orientation module 322 generates and/or processes motion-derived parameters 316 that include rolling averages of one or more accelerometer axis components to monitor device positioning and identify orientation changes. For instance, the orientation module 322 may compute rolling averages of x and y acceleration components over predetermined time intervals, such as 24-hour periods, to establish baseline device orientation patterns and detect deviations that indicate inversion events.

The orientation module 322 may further determine device inversion events by comparing the range of rolling averages against predefined thresholds. For instance, if the range of rolled averages exceeds a threshold value, the orientation module 322 may identify that a device flip or inversion event has occurred. In some implementations, the orientation module 322 leverages the detected inversion events to inform signal processing corrections for other modules within the analysis module 304, such as automatically adjusting ECG signal polarity and/or correcting accelerometer-based features to maintain data integrity throughout extended monitoring sessions. As such, the orientation module 322 is operable to process the accelerometer data 310 to detect device orientation changes and provide inversion event information that can be used to enhance accuracy of other physiological measurements and predictions during the wear period.

The multiparameter module 324 is operable to analyze motion-derived parameters 316 in combination with additional sensor data to generate insights 116 informed by multimodal data sources. In an example, the multiparameter module 324 generates and/or processes motion-derived parameters 316 in addition to ECG data 314 to create comprehensive insights 116 that leverage information from accelerometer-based and cardiac monitoring. For instance, the multiparameter module 324 may combine acceleration magnitude and activity parameters with heart rate variability metrics to distinguish between different types of inactive states, such as to distinguish between states such as “restful sleep” and “sedentary wakefulness” based on movement patterns and cardiac rhythm characteristics.

The multiparameter module 324 may further implement machine learning algorithms (e.g., from the machine learning system 326) trained on multimodal datasets such as to optimize integration of accelerometer and ECG-derived features. For instance, the multiparameter module 324 can apply weighted combination schemes that dynamically adjust a relative importance of accelerometer-based versus ECG-based predictions based on signal quality, user-specific patterns, computational resource consumption, and/or temporal context during the wear period. In some implementations, the multiparameter module 324 leverages insights generated by one or more other modules within the analysis module 304, such as via incorporation of body position information from the position module 320 to enhance accuracy of sleep stage detection by providing contextual information about user posture during various cardiac rhythm patterns. In this way, the multiparameter module 324 is operable to process both the accelerometer data 310 and ECG data 314 to generate multimodal relationship 124 insights to provide robust and clinically relevant predictions.

The analysis module 304 can further leverage the machine learning system 326 to generate one or more of the insights 116. The machine learning system 326, for instance, can apply trained algorithms to generate and/or process the motion-derived parameters 316 and other sensor data to generate accurate user state classifications and physiological insights. The machine learning system 326 may include one or more trained models that have been optimized for processing low sample rate data, such as neural networks, decision trees, support vector machines, or ensemble methods that combine multiple algorithmic approaches.

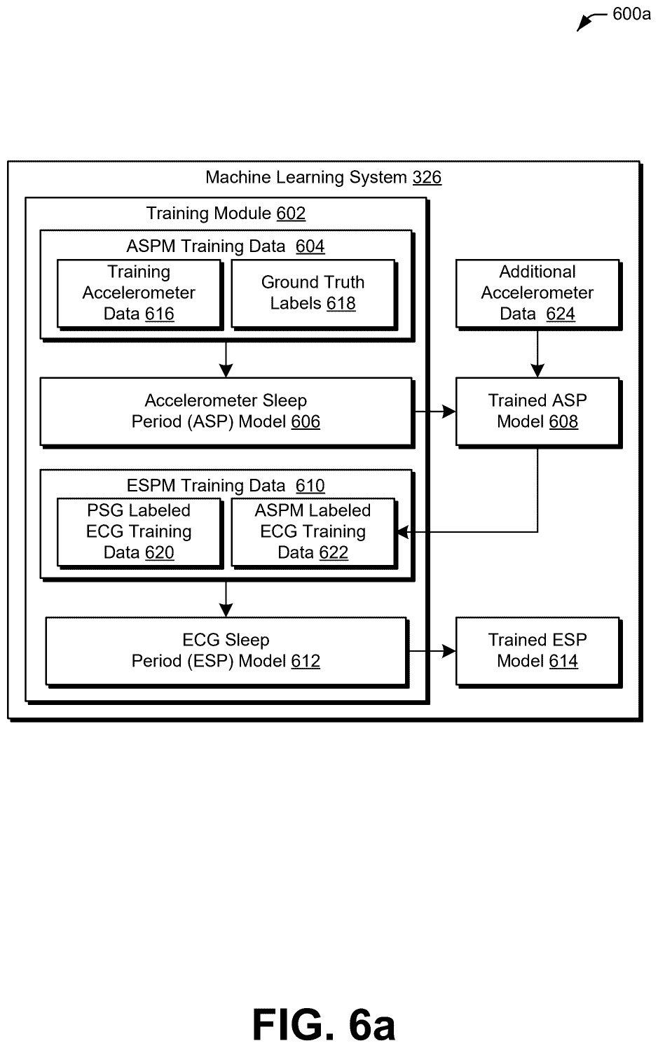

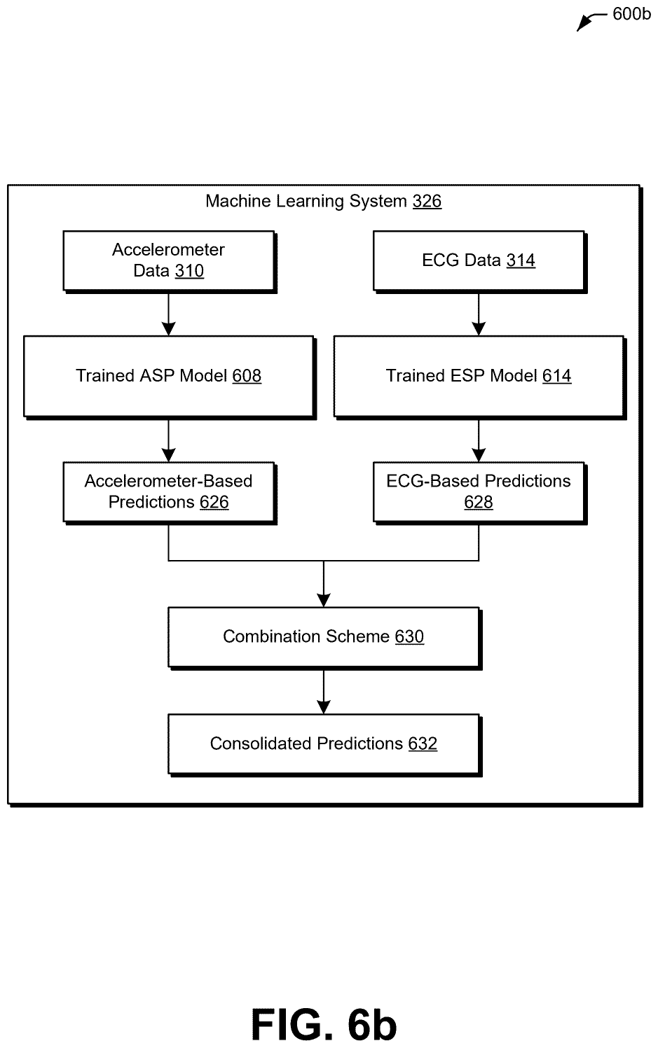

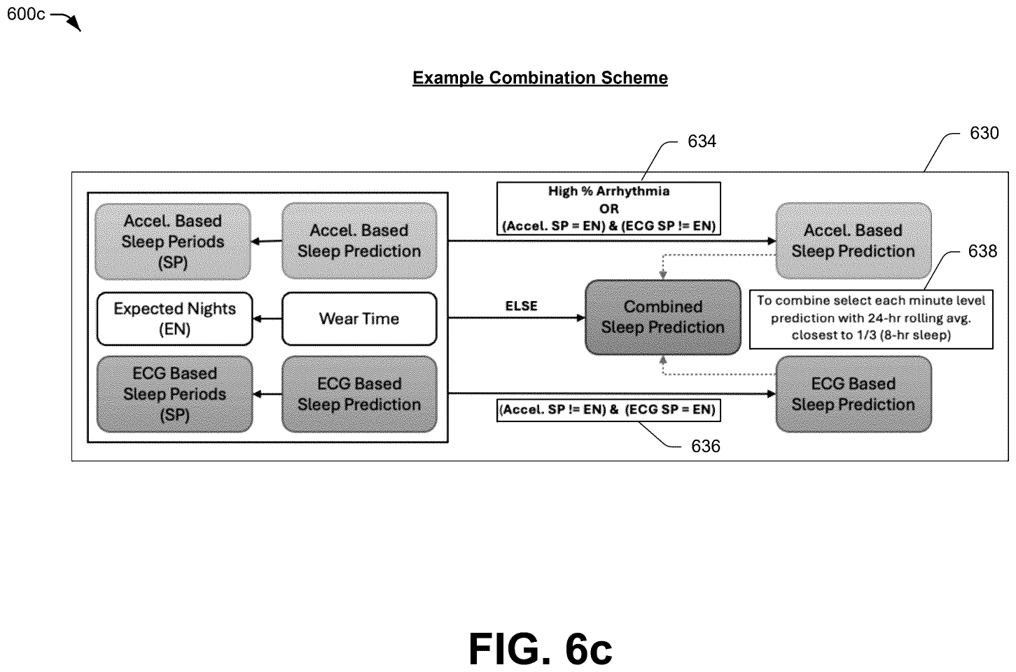

In some implementations, the machine learning system 326 may employ separate models for different prediction tasks, such as dedicated models for sleep detection, activity classification, body position determination, and/or multimodal predictions, which may be trained on domain-specific datasets to enhance accuracy for their respective functions. The machine learning system 326 may also incorporate adaptive learning capabilities that can adjust model parameters based on user-specific patterns observed during the wear period, such as to enable personalized predictions that account for individual variations in movement patterns, sleep behaviors, and physiological responses. Additionally, the machine learning system 326 may implement feature selection and dimensionality reduction techniques to optimize processing efficiency while maintaining prediction accuracy under the constrained computational resources of wearable devices. Additional features and examples the machine learning system 326 are discussed in more detail below, such as with respect to FIGS. 6a and 6b.

Accordingly, the insight 116 can provide a variety of meaningful information about various aspects of the user's physiological state and device performance during the monitoring period. The insight 116 may be generated using various computational approaches, e.g., algorithmic processing, statistical analysis, machine learning techniques, or combinations thereof, and can include both real-time determinations and post-processing analyses. In various implementations, the insight 116 may be presented in different formats such as numerical values, categorical classifications, graphical representations, or textual summaries, and may be tailored for a particular audience such as healthcare providers, researchers, end users, and so forth.

The presentation module 306 is operable to format and configure the insights 116 for output to users, healthcare providers, or other systems. In various implementations, the presentation module 306 may generate visual displays, textual summaries, graphical representations, or interactive interfaces that present the processed physiological data in a clinically meaningful format. The presentation module 306 may also implement customizable reporting features that allow different types of information to be emphasized or filtered based on specific use cases or user preferences.

For instance, the presentation module 306 can generate a report 328 that includes one or more insights 116. The report 328 may be configured in various formats, such as a PDF document, an interactive web-based interface, a mobile application display, or other digital or printed formats suitable for healthcare providers and users. In some implementations, the report 328 may include summary sections that provide aggregate data over the wear period, daily breakdown sections that show temporal patterns, and graphical visualizations that correlate multiple data types such as heart rate patterns overlaid with sleep and activity information.

In some examples, the prediction system 114 is further operable to dynamically adjust device properties or operational parameters based on the generated insights 116. For instance, the prediction system 114 may modify sampling rates of one or more sensors during the wear period in response to detected user states, such as to increase accelerometer sampling frequency during active periods to capture detailed movement patterns or to reduce sampling rates during sleep states to conserve battery power. The prediction system 114 may also trigger activation or deactivation of one or more sensors based on the insights 116. In some implementations, the prediction system 114 may adjust signal processing parameters in real-time and/or implement power management strategies that optimize battery usage by selectively enabling or disabling various device functions based on one or more insights 116.

In various implementations, the processing operations performed by the analysis module 304 may be executed during and/or after the wear period and may be performed synchronously and/or asynchronously. For example, extraction of motion-derived parameters 316 and generation of insights 116 may occur in real-time on the monitoring device 104 during the wear period, such as to enable immediate feedback or triggering of additional sensor operations based on detected user states. Additionally or alternatively, the accelerometer data 310 and/or ECG data 314 may be transmitted wirelessly or via wired connection to one or more external computing devices, such as smartphones, tablets, or server systems for analysis. In at least one example, the accelerometer data 310 and ECG data 314 are stored locally on the monitoring device 104 during the wear period and downloaded upon termination of the wear period.

FIG. 4 depicts a nonlimiting example 400 of physiological monitoring using low sample rate accelerometer data in which insights 116 are generated that include a sleep/activity state 118.

The example 400 includes a first scenario 402 and a second scenario 404 that demonstrate how the analysis module 304 processes accelerometer data 310 collected by a wearable device to generate sleep/activity states 118 based on temporal movement patterns of a user during a wear period. In the first scenario 402, a user is in a recumbent position for approximately fifteen minutes, which represents a period of relatively minimal physical activity, e.g., a resting state. The accelerometer data 310 collected by the monitoring device 104 during this period includes three orthogonal acceleration components (e.g., x, y, and z components) that correspond to respective axes of the monitoring device 104. During the resting state, the accelerometer data 310 remains relatively constant across the three axes, with relatively minor fluctuations due to natural physiological movements such as breathing or slight positional adjustments.

The analysis module 304 processes the accelerometer data 310 from the first scenario 402 to calculate one or more motion-derived parameters 316, such as an acceleration magnitude 406 and an activity parameter 408. The acceleration magnitude 406 may be calculated as a square root of a sum of squares of the individual acceleration components of the low sample rate accelerometer data 310. For instance, given an acceleration vector â=(xₜ, yₜ, zₜ) for a given time t, the acceleration magnitude 406 may be calculated as:

$$\vert {P}_{t} \vert = \sqrt{{x}_{t}^{2} + {y}_{t}^{2} + {z}_{t}^{2}} .$$

For a particular temporal interval 410 of the wear period, the activity parameter 408 may be calculated as a standard deviation (o) of the acceleration magnitude 406 over the temporal interval 410. In the first scenario 402, the relatively constant nature of the accelerometer data 310 results in a low standard deviation value for the activity parameter 408 (e.g., σ<threshold) which corresponds to user stillness. Based on these motion-derived parameters 316, the analysis module 304 generates an insight 116 that indicates that the sleep/activity state 118 corresponds to an inactive state.

In the second scenario 404, the user is engaged in a running activity for a fifteen-minute duration. During this active period, the accelerometer data 310 exhibits significant changes in acceleration values along each axis. The analysis module 304 processed the accelerometer data 310 to calculate the acceleration magnitude 406 and activity parameter 408 as described above.

The variation in the accelerometer data 310 during the running activity results in a relatively elevated standard deviation value (e.g., σ>threshold) for the activity parameter 408 over the temporal interval 410. The relatively high standard deviation corresponds to user movement and indicates substantial physical activity. Accordingly, the analysis module 304 processes such motion-derived parameters 316 to generate an insight 116 that classifies the sleep/activity state 118 as an active state. In this way, the example 400 demonstrates how the prediction system 114 can accurately distinguish between various user states via analysis of motion-derived parameters 316 extracted from low sample rate accelerometer data 310, which supports reliable classification of sleep/activity states 118 under power-constrained conditions.

FIG. 5 depicts a nonlimiting example 500 of physiological monitoring using low sample rate accelerometer data in which body angles and positions are generated based on accelerometer data.

The example 500 illustrates body angle determination based on low sample rate accelerometer data for three different positions of a person 102 wearing a monitoring device 104. The example 500 includes an upright position diagram 502 showing the person 102 wearing the monitoring device 104 in a vertical orientation, a recumbent position diagram 504 showing the person 102 wearing the monitoring device 104 in a horizontal orientation, and an angled position diagram 506 showing the person 102 wearing the monitoring device 104 at approximately 45 degrees. The diagrams depict various vector components, such as a reference vector 508, a first position vector 510, a second position vector 512, and a third position vector 514.

The reference vector 508, for instance, represents a baseline orientation measurement that serves as a coordinate system anchor point for subsequent body angle calculations. In various implementations, the reference vector 508 may be computed based on accelerometer data 310 obtained during periods of high activity, such as walking or other ambulatory movements, and thus corresponds to an “upright” position of the person 102, e.g., 90 degrees. This is by way of example and not limitation, and in various examples the reference vector 508 is computed using statistical analysis of accelerometer data 310 during periods of relatively low activity, such as during a period of sleep, and thus corresponds to a “reclined” position of the person 102, e.g., zero degrees.

The reference vector 508 may be determined using various statistical analyses, such as using machine learning algorithms, predetermined calibration procedures, or other computational approaches that identify when the user is most likely in a particular orientation/body angle. By way of example and not limitation, the reference vector 508 may be calculated as an average of acceleration measurements during periods of the monitoring session that are above a threshold level of activity and/or indicate vertical positioning. In various examples, one or more components of the accelerometer data 310 can be weighted during calculation of the reference vector 508, such as to emphasize one or more axes or directional components that are relatively more indicative of upright positioning during high activity periods, thereby improving the accuracy of body angle calculations throughout the wear period.

To determine a body angle of the person 102 at a particular temporal instance, the prediction system 114 calculates a position vector that represents an orientation of the monitoring device 104 based on the three-dimensional accelerometer data components at the particular temporal instance. The position vector, for instance, can be derived from the raw accelerometer measurements by processing the x, y, and z acceleration components to create a directional vector that indicates a spatial orientation of the device. The prediction system 114 is then able to determine a body angle based on a polar angle in a spherical coordinate system between the reference vector 508 and a particular position vector.

For instance, in an example in which the reference vector 508 represents an upright position, values of the polar angle can range from 0 degrees for substantially upright positions to 90 degrees for substantially recumbent positions. In the illustrated example, the polar angle (q) is approximately 0 degrees in the upright position diagram 502, 90 degrees in the recumbent position diagram 504, and 45 degrees in the angled position diagram 506.

Further, the prediction system 114 is able to generate classifications of user body position 120 based on the determined body angle. By way of example and not limitation, the prediction system 114 may categorize body positions 120 into discrete states such as upright, seated, reclining, or supine orientations based on predetermined angle thresholds. For instance, in the upright position diagram 502 the body angle is approximately 0 degrees, and the prediction system 114 determines the person 102 is in a standing or vertical posture. In the recumbent position diagram 504 the body angle is approximately 90 degrees, and accordingly the prediction system 114 determines the person 102 is in a lying down or horizontal posture. In the angled position diagram 506, the body angle is approximately 45 degrees, and thus the prediction system 114 determines the person 102 is in an intermediate position between upright and recumbent orientations.

FIGS. 6a, 6b, and 6c depict nonlimiting examples 600a, 600b, and 600c of physiological monitoring using low sample rate accelerometer data in which a machine learning system 326 is trained, configured, and implemented to generate insights 116 based on various parameters.

In the example 600a, the machine learning system 326 is configured to train one or more models to process low sample rate accelerometer data and/or ECG data to generate insights 116. As further described in more detail below, the machine learning system 326 can include and/or is representative of one or more types of machine learning model, such as but not limited to neural networks, decision trees, support vector machines, random forests, ensemble methods, convolutional neural networks, recurrent neural networks, long short-term memory networks, gradient boosting algorithms, logistic regression models, k-nearest neighbor classifiers, and so forth.

To begin in this example, the machine learning system 326 includes a training module 602 that receives various types of training data and applies machine learning techniques to develop/train models to process various data modalities to generate insight 116. For instance, the training module 602 utilizes accelerometer sleep period model (“ASPM”) training data 604 to train an accelerometer sleep period (“ASP”) model 606, such as to generate a trained ASP model 608. The training module 602 is further operable to use ECG sleep period model (“ESPM”) training data 610 to train an ESP model 612 to generate a trained ESP model 614. Such training may happen synchronously, asynchronously, in parallel, sequentially, and/or using various other temporal schema depending on computational resources and system requirements.

The ASPM training data 604 can include training accelerometer data 616 that represents low sample rate accelerometer measurements collected from wearable devices during previous monitoring periods. The ASPM training data 604 also includes ground truth labels 618 that correspond to verified conditions during time periods when the training accelerometer data 616 was collected. The ground truth labels 618 may include various conditions of the wear period, such as sleep states, active states, inactive states, body positions, device orientations, and so forth.

The training module 602 processes the training accelerometer data 616 and the ground truth labels 618 to train the ASP model 606 to generate insights 116 based on low sample rate accelerometer measurements. The training process may employ various machine learning techniques to adjust one or more weights and/or learn one or more parameters of the model 606 as part of the training. In some implementations, the training module 602 may utilize cross-validation techniques, feature selection algorithms, or data augmentation methods such as to enhance model performance and generalization capabilities across diverse user populations and monitoring conditions.

The training module 602 also utilizes ESPM training data 610 to train the ESP model 612, such as to generate the trained ESP model 614. The ESPM training data 610, for instance, includes ECG measurements with corresponding labels, e.g., sleep and activity labels, that are used to train the ESP model 612 to generate predictions. In the illustrated example, the ESPM training data 610 includes PSG labeled ECG training data 620 as well as ASPM labeled ECG training data 622.

The PSG labeled ECG training data 620, for instance, represents ECG data collected during polysomnography (“PSG”) studies with corresponding sleep stage labels that are validated, e.g., through clinical sleep monitoring protocols. The ASPM labeled ECG training data 622 represents ECG measurements that have been labeled using predictions generated by the trained ASP model 608. For example, the machine learning system 326 leverages the trained ASP model 608 to process additional accelerometer data 624 collected along with corresponding ECG data to generate labels, e.g., sleep and activity labels, that are then applied to the corresponding ECG data. In this way, incorporation of the ASPM labeled ECG training data 622 in the ESPM training data 610 ensures that wake periods are adequately represented during training of the ESP model 612.

The training module 602 processes the PSG labeled ECG training data 620 and the ASPM labeled ECG training data 622 to train the ESP model 612 to generate insights 116 based on collected ECG data 314. The training process may involve iteratively adjusting model parameters, such as neural network weights, decision tree thresholds, or support vector machine hyperparameters, to minimize prediction errors and optimize performance on the training datasets. Various optimization techniques may be employed during training, including gradient descent algorithms, backpropagation methods, regularization approaches, or ensemble learning strategies that combine multiple models to enhance prediction accuracy and robustness across different user populations and monitoring scenarios.

Referring to FIG. 6b, the example 600b depicts implementation of the trained ASP model 608 and the trained ESP model 614 to process input data. For instance, the trained ASP model 608 receives and processes accelerometer data 310 to generate accelerometer-based predictions 626, such as by analyzing motion-derived parameters extracted from the accelerometer data 310 to identify patterns associated with sleep states, active states, and inactive states of a user during a wear period. The trained ESP model 614 receives and processes ECG data 314 to generate ECG predictions 628. The trained ESP model 614, for instance, can detect sleep states and/or stages by analyzing heart rate variability, cardiac rhythm patterns, and other physiological indicators present in the ECG data 314.

The machine learning system 326 is operable to combine the accelerometer-based predictions 626 and the ECG-based predictions 628 in accordance with a combination scheme 630 to generate consolidated predictions 632. The combination scheme 630, for instance, represents a framework that integrates the predictions to enhance accuracy and reliability of the insights 116. For example, the combination scheme 630 may select between predictions generated by the trained ASP model 608 and the trained ESP model 614 and/or may combine two or more predictions based on various considerations. An example combination scheme 630 is depicted in FIG. 6c and further discussed in more detail below.

Accordingly, the consolidated predictions 632 can include values from either or both the accelerometer-based predictions 626 and/or the ECG-based predictions 628. In one or more examples, the consolidated predictions 632 include insights 116 for a duration of the wear period, such as minute-level classifications of user states such as sleep, active, and inactive periods. In some examples, the consolidated predictions 632 may incorporate body position information and/or device orientation predictions.

Referring to FIG. 6c, the example 600c illustrates a combination scheme 630 that the machine learning system 326 leverages to integrate accelerometer-based predictions 626 with ECG-based predictions 628, such as by using heuristic decision logic to generate the consolidated predictions 632. For instance, the combination scheme 630 determines whether to use a particular accelerometer-based prediction 626, a particular ECG-based prediction 628, or a combination of the two for a particular temporal interval, e.g., for a minute interval of the wear period. The combination scheme 630, for instance, can be leveraged to determine minute level predictions for the duration of the wear period. The combination scheme 630 can determine which predictions to include in the consolidated predictions 632 based on considerations such as signal quality, prediction confidence levels, consistency with expected physiological patterns, and so forth.

By way of example, the machine learning system 326 is operable to identify irregular conditions of the accelerometer-based predictions 626 and/or the ECG-based predictions 628 that may be inaccurate. Such irregular conditions may include but are not limited to instances in which the ECG-based predictions 628 (and/or the ECG data 314) indicate arrhythmia above a threshold level, which can interfere with ECG-based sleep detection algorithms. Additionally or alternatively, irregular conditions may arise when accelerometer-derived sleep/wake patterns deviate above a significance threshold from expected patterns. Further, such irregular conditions may occur when ECG-based sleep/wake patterns deviate above a significance threshold from expected patterns, which may suggest cardiac irregularities or signal quality degradation that impacts accuracy.

The machine learning system 326 leverages the combination scheme 630 to reconcile such irregular conditions. For example, as shown at a first node 634, if the machine learning system 326 detects that an arrhythmia percentage of the ECG data 314 for a particular temporal period is above a threshold, the combination scheme 630 selects the accelerometer-based prediction 626 to include in the consolidated predictions 632. As further shown at the first node 634, if the accelerometer-based prediction 626 and the ECG-based predictions 628 both deviate above a significance threshold from an expected value, the combination scheme 630 selects the accelerometer-based prediction 626. This may be because the accelerometer data 310 is relatively less susceptible to interference due to cardiac arrhythmia than the ECG data 314.

As shown at a second node 636, if the accelerometer-based prediction 626 deviates above a significance threshold from expected nightly patterns while the ECG-based prediction 628 remains within an acceptable range, the combination scheme 630 selects the ECG-based prediction 628 to include in the consolidated predictions 632. This may occur when device displacement, unusual user movement patterns, or accelerometer sensor issues compromise motion-based sleep detection while cardiac rhythm patterns remain stable and reliable.

As shown at a third node 638, if the conditions of the first node 634 or the second node 636 are not met, the combination scheme 630 can select a prediction using alternative criteria. In some examples, this may include combination of the accelerometer-based predictions 626 and the ECG-based predictions 628, such as by using a weighting scheme to average two or more values for a particular interval. Additionally or alternatively, the combination scheme 630 can select a prediction based on whichever modality produces a rolling average, e.g., a 24-hour rolling average that is “closest” to a target, e.g., ⅓, which represents approximately 8 hours of sleep per day. For instance, the combination scheme 630 may select the accelerometer-based prediction 626 if a respective 24-hour rolling average indicates 7.8 hours of sleep while the ECG-based prediction 628 indicates 5.2 hours of sleep, as the accelerometer-based predictions 626 is relatively closer to the target of 8 hours per day.

Accordingly, the machine learning system 326 provides a robust framework for generating accurate insights 116 by leveraging specialized training approaches to process multimodal data. Further, implementation of combination schemes 630 that dynamically select and/or integrate predictions based on likelihoods of signal quality enhance reliability of sleep and activity classifications under various monitoring conditions. In this way, the system can maintain high accuracy under constrained power conditions, which supports extended wear periods without compromising prediction quality.

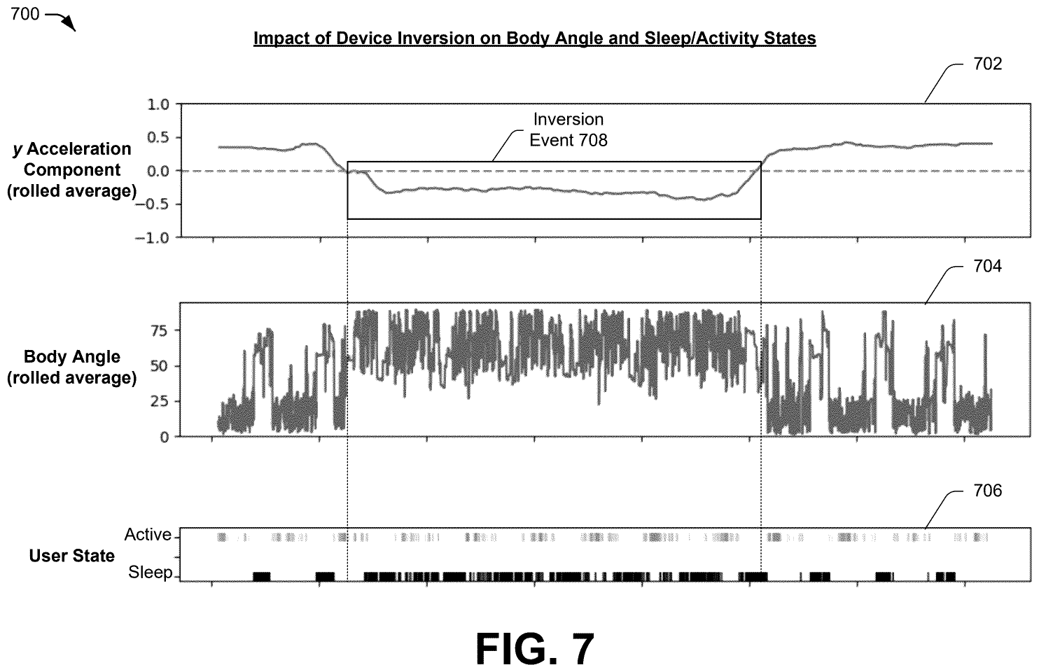

FIG. 7 depicts a nonlimiting example 700 of physiological monitoring using low sample rate accelerometer data in which device inversion can cause measurement inaccuracies.

The example 700 depicts an acceleration component graph 702, a body angle graph 704, and a user state graph 706. The acceleration component graph 702 represents a y component of the accelerometer data 310 as a rolled average over time. The acceleration component graph 702 further depicts an inversion event 708, which is visually represented where values of the y component drop below zero.

As a result of the inversion event 708, the body angle graph 704 demonstrates that when the monitoring device 104 is inverted, body angle calculations may become inaccurate. For instance, this is because a reference frame to determine a user position relative to gravity has changed. The user state graph 706, which indicates sleep/activity states throughout the monitoring period, further illustrates how predictions of sleep and activity states may be impacted when device inversion occurs. For instance, in various embodiments sleep/activity states 118 are based in part on body position 120, and thus a device inversion event 708 can cause incorrect insights related to sleep/activity states 118. Accordingly, as described in more detail in the following example, the prediction system 114 can detect and account for the inversion event 708 during generation of the insight 116.

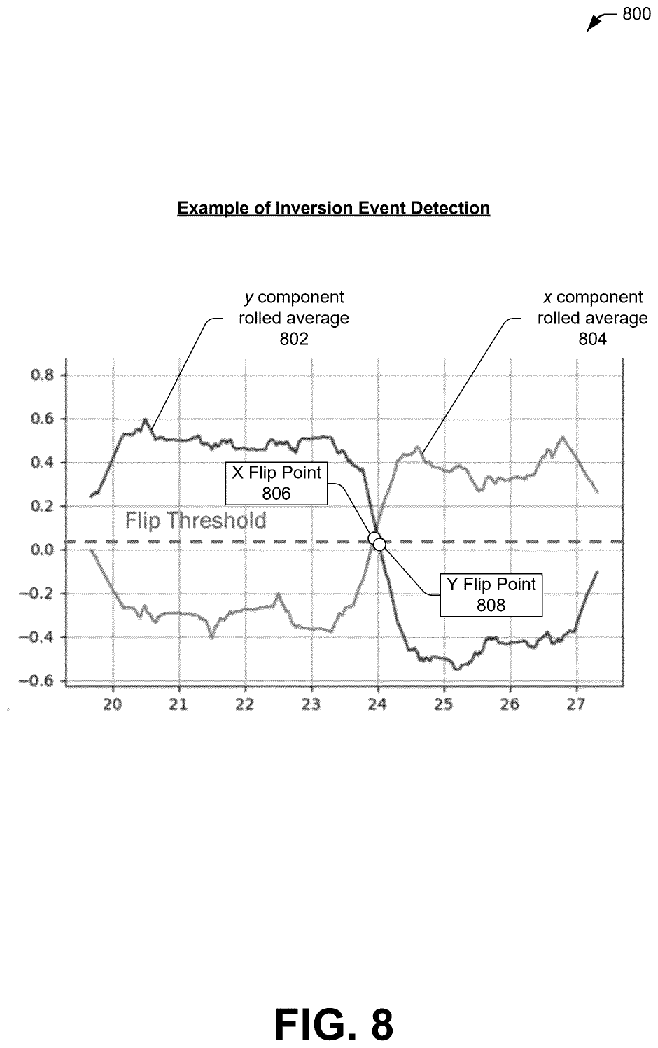

FIG. 8 depicts a nonlimiting example 800 of physiological monitoring using low sample rate accelerometer data including detection of device inversion events.

In this example 800, the prediction system 114 calculates and analyzes rolling averages of one or more acceleration components of the accelerometer data 310 to identify when a wearable device has been flipped or inverted during a monitoring period. For instance, the prediction system 114 analyzes a y component rolled average 802 and an x component rolled average 804, which are illustrated as plotted over time, to detect device orientation changes. The prediction system 114 leverages the rolled averages (such as over a 24-hour period) to smooth short-term variations while preserving long-term trends that correspond to device orientation.

If values of the y component rolled average 802 and/or the x component rolled average 804 exceed one or more thresholds, the prediction system 114 can determine that an inversion event has occurred. In at least one example, the prediction system 114 determines if a range of the y component rolled average 802 and/or the x component rolled average 804 exceeds 0.8 that an inversion event has occurred. The prediction system 114 can further identify an x flip point 806 and/or a y flip point 808 where the respective acceleration components cross predetermined threshold values, which is depicted in the illustrated example.

The prediction system 114 is thus operable to determine a number and timing of inversion events that occur throughout a wear period. This may be output by the prediction system 114, such as included in the report 328 to provide healthcare providers with context about device positioning during the monitoring period. This information may further be used to automatically adjust signal processing parameters for other physiological measurements, such as ECG polarity correction and body angle recalibration to maintain data integrity throughout the wear period.

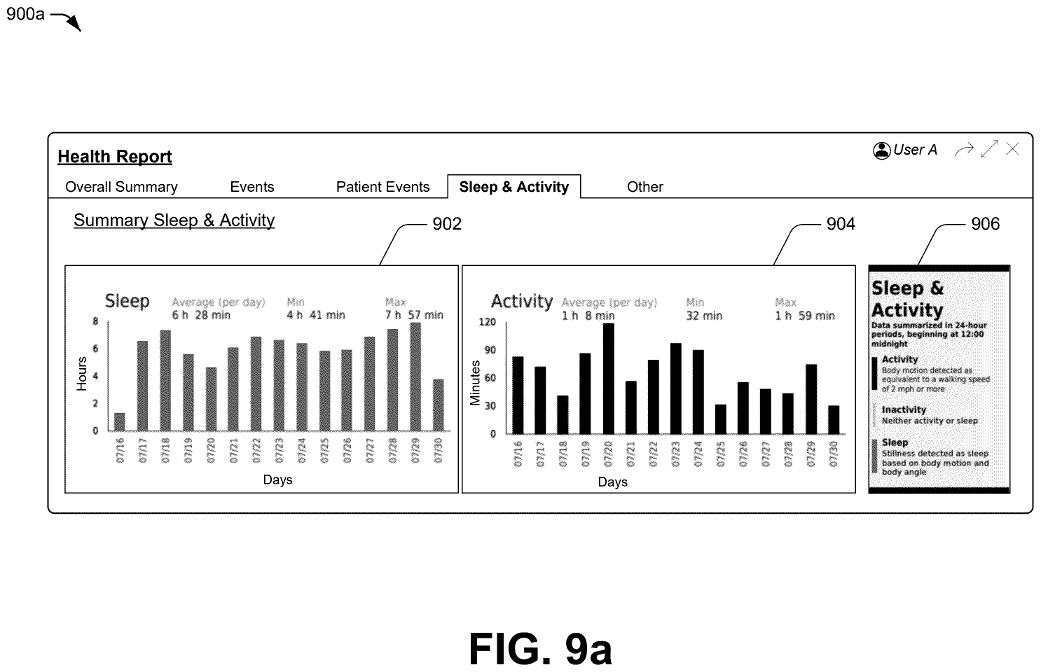

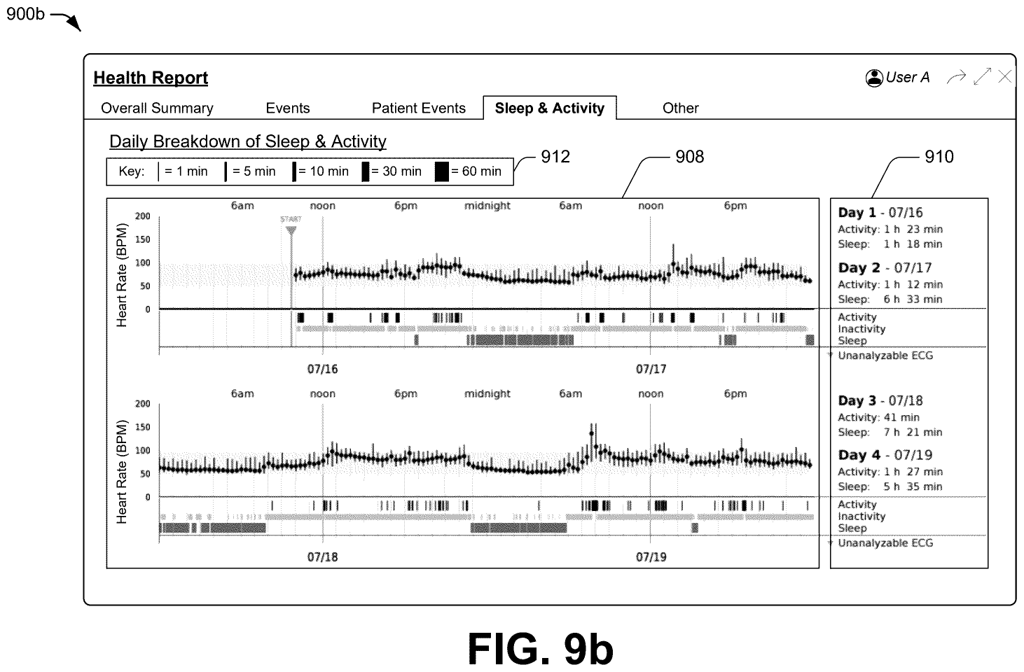

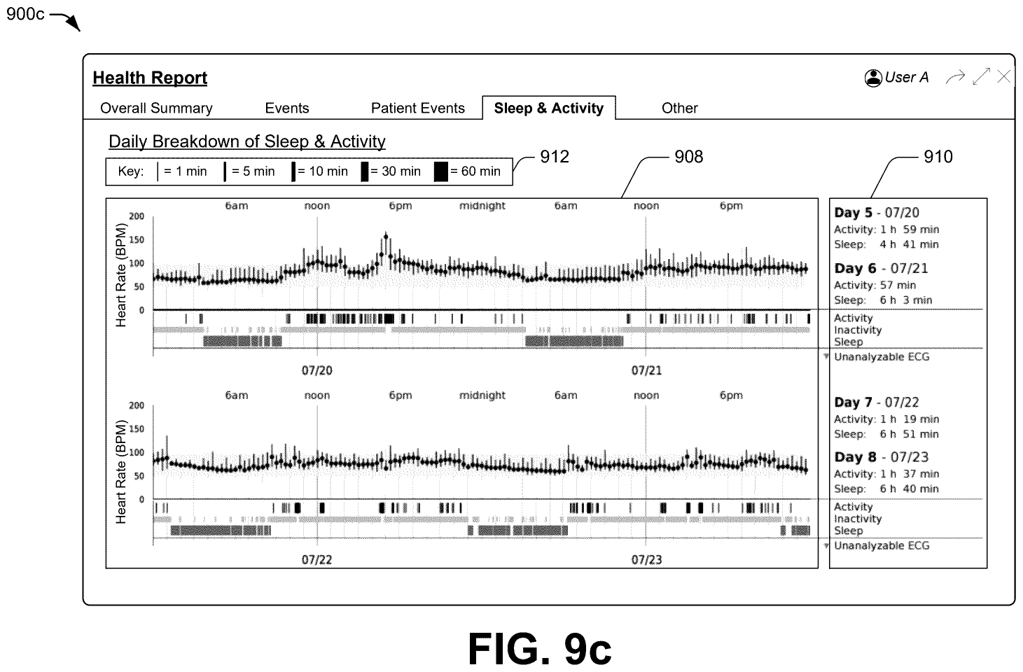

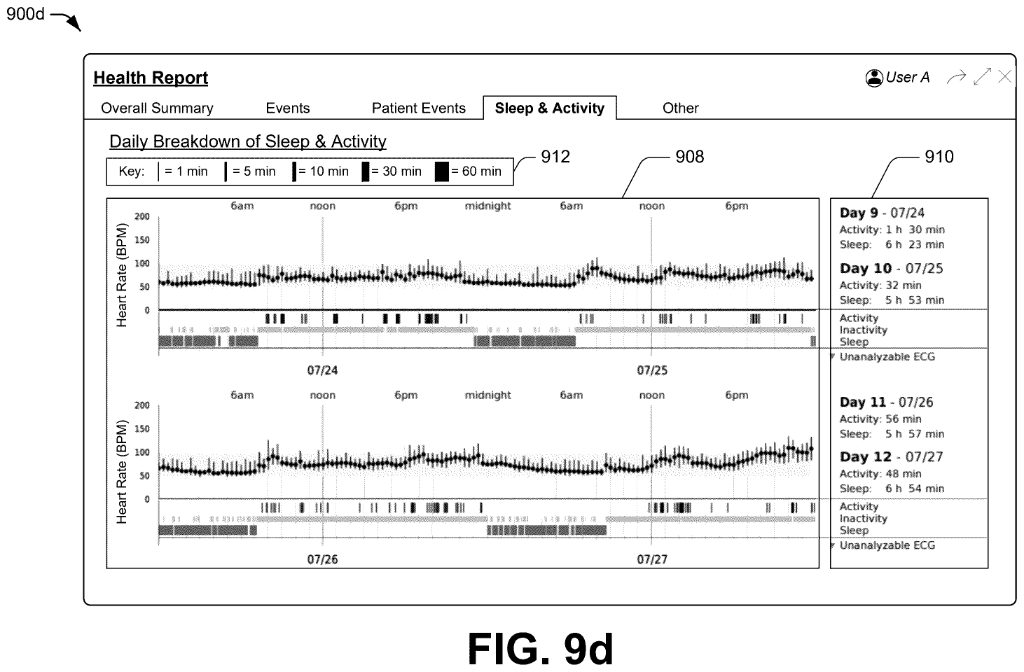

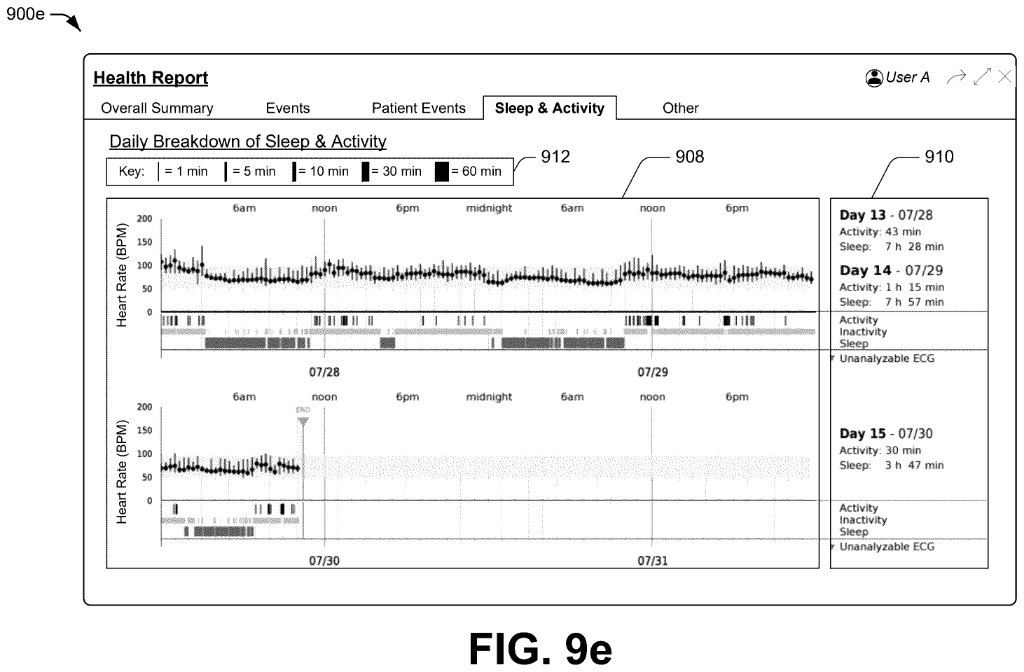

FIGS. 9a-9e depict non-limiting examples of physiological monitoring using low sample rate accelerometer data in which in which a report that includes various insights is output in health report interfaces 900a-900e.

Referring to FIG. 9a a health report interface 900a displays comprehensive sleep and activity monitoring data collected during a wear period. For instance, the health report interface 900a represents a summary section that depicts aggregate user state data for the wear period. For instance, the health report interface 900a includes a sleep data graph 902 that presents sleep duration measurements for various days of the wear period. An activity data graph 904 shows corresponding activity duration measurements for the same temporal period. The sleep data graph 902 and the activity data graph 904 further include statistics such as daily averages, minimums, and maxima.

The health report interface 900a includes a legend panel 906 that provides contextual information about data summarization methods, including definitions for activity states, inactivity periods, and sleep classifications. The legend panel 906 further includes color coding information, such as to indicate visual representations for activity, inactivity, and sleep that may appear in the health report interfaces 900b-900e.

The health report interfaces 900b-900e, for instance, represent daily breakdown sections that provide granular insights for each day of the wear period, e.g., day one through day fourteen. In one or more examples, the health report interfaces 900b-900e may be included in a report 328 with the health report interface 900a, such as in a single viewable document, PDF, etc. Thus, the health report interfaces 900b-900e are viewable in a user interface such as by “scrolling down”. Additionally or alternatively, one or more of the health report interfaces 900b-900e represent separate pages that are accessible via one or more selectable indicia, e.g., page links, dropdown arrows, and so forth.

The health report interfaces 900b-900e shown in FIGS. 9b-9e each include a daily report section 908, a daily summary section 910, and a key 912 that includes representations of time periods to interpret the daily report section 908. The daily report section 908 depicts cardiac measurements (e.g., heart rate in beats per minute) plotted over time with corresponding activity indicators positioned below the heart rate data. The daily report sections 908 across the various interfaces demonstrate how heart rate data can be overlaid on sleep and activity data to provide healthcare providers with detailed insights into user physiological responses during different behavioral states.

The daily summary section 910 includes aggregate sleep and activity metrics for each respective monitored day. The daily summary section 910 may include summaries of sleep duration, activity time, and inactivity time for each day. In the illustrated example, the daily summary section 910 includes a total sleep duration and a total activity duration for a respective day. The health report interfaces 900a through 900e collectively provide comprehensive visualization tools to output various insights 116, which enables healthcare providers and users to analyze patterns and trends in patient behavior and physiological responses over extended monitoring periods which supports informed clinical decision-making and personalized health assessments.

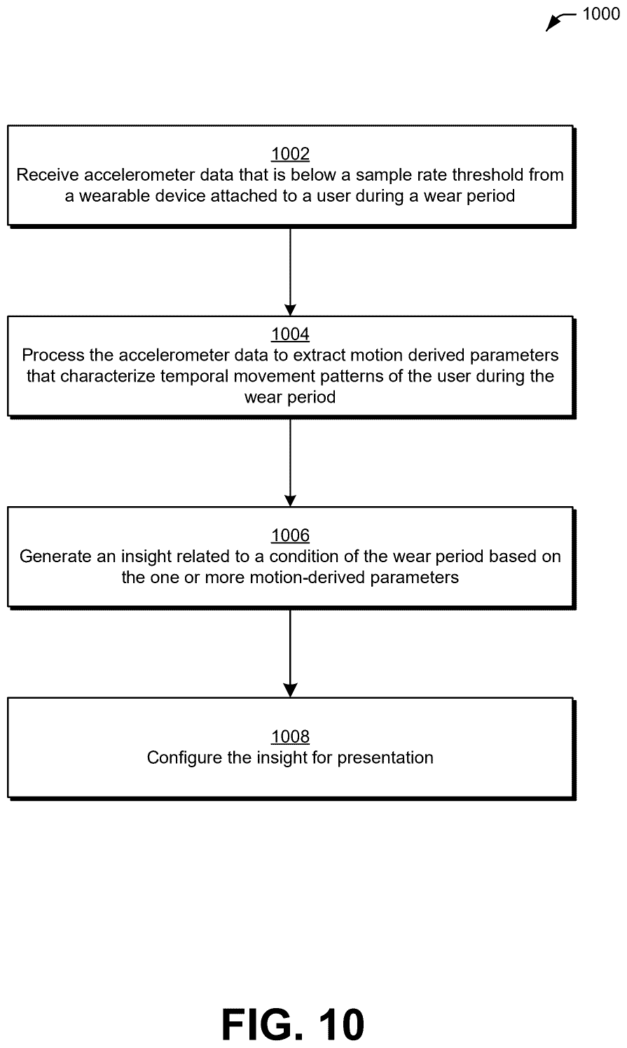

FIG. 10 depicts a flow diagram depicting an algorithm as a step-by-step procedure 1000 in an example implementation that is performable by a processing device to generate insights related to conditions of wear periods based on low sample rate accelerometer data from a wearable device.

To begin in this example, low sample rate accelerometer data having a sample rate that is below a sample rate threshold is received from a wearable device attached to a user during a wear period (block 1002). The accelerometer data, for instance, is produced by the monitoring device 104 during the wear period. In various examples, the monitoring device 104 detects motion and orientation changes of the person 102 using the accelerometer 308 and produces the accelerometer data 310 based on detected movement patterns. In at least one example, the sample rate is approximately 1.56 Hz and the wear period may extend from one to fourteen days.

The low sample rate accelerometer data is then processed to extract one or more motion-derived parameters that characterize temporal movement patterns of the user during the wear period (block 1004). The motion-derived parameters 316, for instance, are computed by the prediction system 114 to quantify various aspects of user movement and positioning. For example, the analysis module 304 calculates acceleration magnitude, activity parameters, reference and/or position vectors for body angle calculations, rolling averages of accelerometer axis components for device orientation detection, and so forth.

An insight related to a condition of the wear period is then generated based on the one or more motion-derived parameters (block 1006). The insight 116, for instance, is produced by the prediction system 114 through analysis of the extracted motion-derived parameters. In various examples, the insight 116 includes predictions of sleep states, active states, and inactive states of the user during temporal intervals of the wear period. The insight 116 may also encompass body angle measurements and/or body position classifications that indicate whether the user is upright, reclining, or lying down at particular times. Additionally or alternatively, the insight 116 can include detection of inversion events of the wearable device during the wear period. In some embodiments, the prediction system 114 implements a machine learning system 326 to process the motion-derived parameters using trained machine learning algorithms/models configured to analyze low sample rate accelerometer data.

The insight is further configured for presentation (block 1008). For instance, the prediction system 114 can format the insight 116 into a user interface display that can be viewed by healthcare providers or users. In various examples, the prediction system 114 creates a comprehensive report that includes a summary section depicting aggregate user state data for the wear period and a daily breakdown section that depicts the user state in temporal correlation with physiological data collected during the wear period. The report 328 may overlay heart rate data with sleep and activity information to provide a holistic view of user health patterns. The presentation may also include graphical representations of body angle changes over time, activity level variations, and detected device orientation events that occurred during monitoring.

The prediction system 114 can further perform a variety of functionality based on the insight 116, such as adjusting device operational parameters during the wear period, triggering additional sensor measurements based on detected user states, generating alerts or notifications when specific conditions are identified, correlating the insight 116 with other physiological data streams, automatically calibrating signal processing algorithms based on the insight 116, providing real-time feedback to users, storing insights 116 for long term analysis, transmitting insights 116 to healthcare providers for remote monitoring, modifying data collection strategies based on detected user behaviors, integrating insights with electronic health records for comprehensive patient assessment, and so forth.

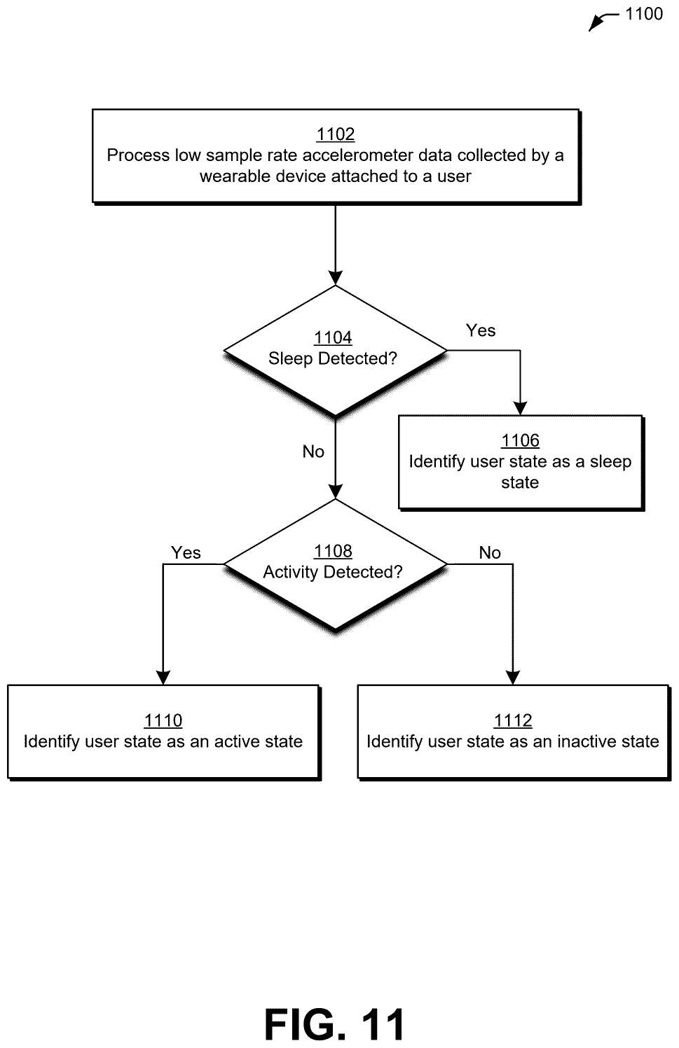

FIG. 11 depicts a flow diagram depicting an algorithm as a step-by-step procedure 1100 in an example implementation that is performable by a processing device to generate insights related to user states based on low sample rate accelerometer data. The procedure 1100, for instance, can be implemented as one or more substeps of one or more of the preceding or subsequent flow diagrams. In various example, the procedure 1100 represents a decision tree logic implemented by an algorithm that sequentially evaluates motion-derived parameters to determine whether a user is in a sleep state, active state, or inactive state during a wear period.

To begin in this example, low sample rate accelerometer data collected by a wearable device attached to a user is processed (block 1102). The low sample rate accelerometer data, for instance, may be collected at approximately 1.56 Hz by the monitoring device and is processed to extract motion-derived parameters 316 that characterize temporal movement patterns of the user during the wear period. The motion-derived parameters 316 may include an acceleration magnitude 406 calculated as a square root of a sum of squares of one or more acceleration components of the low sample rate accelerometer data and/or an activity parameter 408 calculated as a standard deviation of the acceleration magnitude. For instance, a relatively low standard deviation may correspond to user stillness and a relatively high standard deviation may correspond to user movement.

A determination is then made as to whether sleep is detected based on the processed accelerometer data (block 1104). The sleep detection evaluation may involve analysis of stillness patterns and body angle information derived from the motion-derived parameters 316. In some cases, the analysis module 304 applies threshold comparisons to the activity parameter 408, where low standard deviation values of acceleration magnitude 406 over temporal intervals 410 may indicate user stillness consistent with sleep states. The sleep detection may also incorporate body position 120 information, where reclined body angles approaching 90 degrees relative to an upright reference vector may support sleep state classification.

If sleep is detected (e.g., “Yes” at block 1104), the user state is identified as a sleep state (block 1106). The sleep/activity state 118 classification as a sleep state may be recorded for a particular temporal interval and incorporated into an insight 116 generated by the prediction system 114. In various examples, the sleep state identification may trigger additional processing operations, such as one or more additional sensors and/or adjusting data collection parameters during the detected sleep period.

If sleep is not detected (e.g., “No” at block 1104), the prediction system 114 proceeds to evaluate whether activity is detected (block 1108). The activity detection evaluation may involve comparison of the motion-derived parameters 316 against activity thresholds, such to identify walking speeds of approximately 2 mph or greater. In some examples, the analysis module 304 examines the standard deviation of acceleration magnitude 406 over one or more temporal intervals 410, where relatively high standard deviation values indicate user movement consistent with active states. Additionally or alternatively, the activity detection may be based in part on a body position 120, such that an upright state may indicate activity while a recumbent position may indicate inactivity.

If activity is detected (e.g., “Yes” at block 1108), the user state is identified as an active state (block 1110). The active state classification may be incorporated into the sleep/activity state 118 determination and recorded as part of the insight 116 for the corresponding temporal interval. The active state identification may also influence subsequent processing operations, such as body position 120 calculations or device orientation 122 assessments that may be affected by user movement patterns.

If activity is not detected (e.g., “No” at block 1108), the user state is identified as an inactive state (block 1112). The inactive state, for instance, represents periods where the user is awake but not engaged in movement that meets activity threshold criteria. In various examples, the inactive state classification may correspond to sedentary behaviors such as sitting or reclining without stillness patterns that may be characteristic of sleep states. Thus, the techniques described herein support comprehensive user state categorization throughout the wear period based on the sequential evaluation of motion-derived parameters 316 extracted from low sample rate accelerometer data.

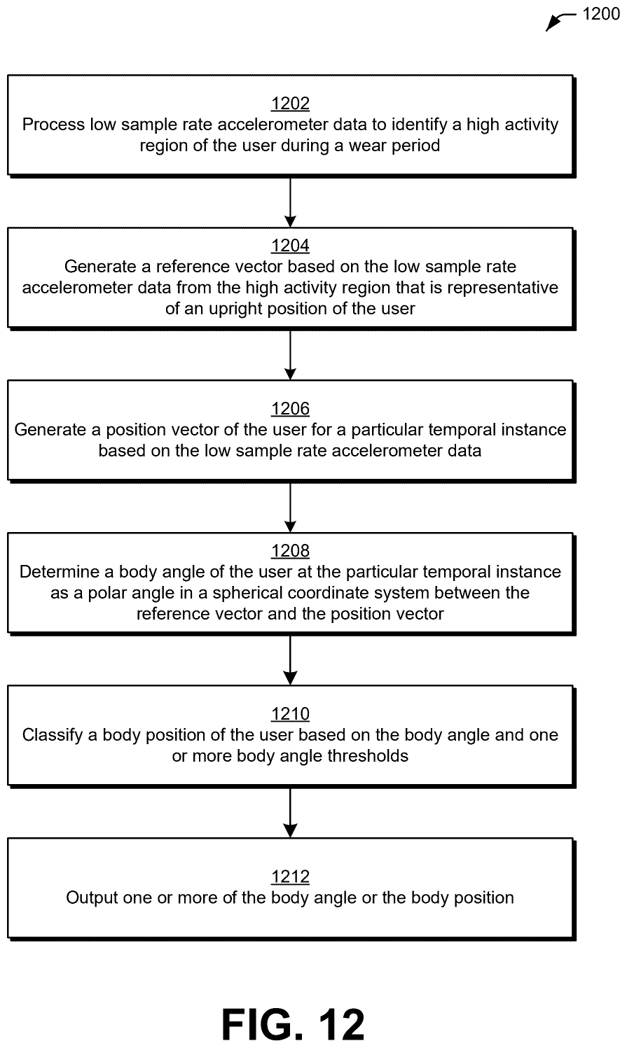

FIG. 12 depicts a flow diagram depicting an algorithm as a step-by-step procedure 1200 in an example implementation that is performable by a processing device to determine body angle and position based on low sample rate accelerometer data collected during extended wear periods. The procedure 1200, for instance, can be implemented as one or more substeps of one or more of the preceding or subsequent flow diagrams.

To begin in this example, low sample rate accelerometer data is processed to identify a high activity region of the user during a wear period (block 1202). The high activity region, for instance, corresponds to periods when the person 102 is engaged in movement patterns that indicate an upright posture or active state. The identification of high activity regions may involve analysis of acceleration magnitude variations, frequency domain characteristics, and/or statistical measures of the accelerometer data 310 over defined time windows.

A reference vector is generated based on the low sample rate accelerometer data from the high activity region that is representative of an upright position of the user (block 1204). The reference vector 508, for instance, serves as a baseline orientation marker against which subsequent body angle calculations are performed. In various examples, the position module 320 computes the reference vector 508 by averaging or processing one or more acceleration components of the accelerometer data 310 from the identified high activity periods to establish a consistent upright reference frame. The reference vector 508 may be calculated using one or more statistical methods such as mean vector computation, principal component analysis, or weighted averaging techniques.

A position vector of the user is generated for a particular temporal instance based on the low sample rate accelerometer data (block 1206). The position vector 510, for instance, represents an orientation of the monitoring device 104 and, by extension, the body position of the person 102 at a particular moment during the wear period. In various examples, the analysis module 304 processes the accelerometer data 310 to extract three-dimensional acceleration components that define the position vector 510 for each temporal sampling point, e.g., every minute. In some examples, multiple position vectors 510 are computed across sequential temporal instances to enable continuous monitoring of body position changes throughout the wear period.

A body angle of the user at the particular temporal instance can then be determined as a polar angle in a spherical coordinate system between the reference vector and the position vector (block 1208). The body angle calculation, for instance, quantifies an angular deviation from the established upright reference to provide a numerical measure of body inclination. The body angle determination may range from 0 degrees for fully upright positioning to 90 degrees for substantially recumbent positioning, such as to provide a continuous measure of postural orientation.

A body position of the user is then classified based on the body angle and one or more body angle thresholds (block 1210). The body position classification, for instance, translates a body angle measurements into a discrete postural category. In various examples, the position module 320 applies predetermined threshold values to categorize body angles into classifications such as upright, sitting, reclining, lying down positions, and so forth. The classification process may utilize multiple threshold boundaries to create distinct ranges for different postural states, enabling differentiation between subtle variations in body positioning.

Once generated, the body angle and/or the body position are output (block 1212). In various examples, the presentation module 306 formats the body angle and position data for inclusion in the report 328 alongside other insights 116 such as sleep/activity state 118 information. The output may include temporal sequences of body position classifications, statistical summaries of postural patterns, or correlations between body positioning and other monitored parameters.

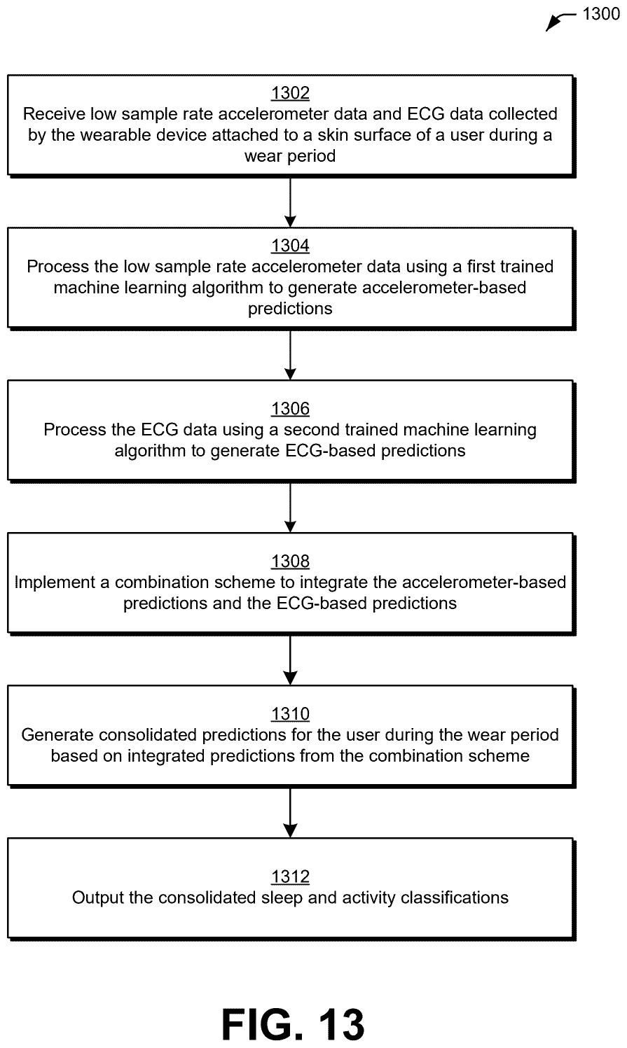

FIG. 13 depicts a flow diagram depicting an algorithm as a step-by-step procedure 1300 in an example implementation that is performable by a processing device to integrate ECG and accelerometer data for enhanced sleep and activity prediction using machine learning. The procedure 1300, for instance, can be implemented as one or more substeps of one or more of the preceding or subsequent flow diagrams. The procedure 1300 demonstrates how separate trained machine learning algorithms can process multimodal data to generate consolidated classifications.

To begin in this example, low sample rate accelerometer data and ECG data collected by the wearable device attached to a skin surface of a user during a wear period are received (block 1302). In various examples, the monitoring device 104 detects motion patterns using the accelerometer 308 and detects electrical activity of the heart using the ECG sensor 312 to produce the accelerometer data 310 and ECG data 314.

The low sample rate accelerometer data is processed using a first trained machine learning algorithm to generate accelerometer-based predictions (block 1304). The first trained machine learning algorithm, for instance is the trained ASP model 608 that is configured to analyze motion-derived parameters 316 extracted from the accelerometer data 310 to generate predictions, e.g., to classify user states.

The ECG data is processed using a second trained machine learning algorithm to generate ECG-based predictions (block 1306). The second trained machine learning algorithm, for instance, is the trained ESP model 614 that is configured to analyze cardiac parameters to generate predications, e.g., to classify user states. The second trained machine learning algorithm may be trained using a combination of polysomnography labeled ECG training data 620 and ASPM labeled ECG training data 622 such as to ensure representation of both sleep and wake periods in the training set.

A combination scheme is implemented to integrate the accelerometer-based predictions and the ECG-based predictions (block 1308). The combination scheme 630, for instance, applies heuristic methods to determine which prediction source to use for various temporal intervals of the wear period. In some cases, the combination scheme 630 evaluates factors such as arrhythmia prevalence, signal quality, and consistency between the two prediction sources to make integration determinations. Alternatively or additionally, the combination scheme 630 may combine predictions generated by the first and second machine learning algorithms, such as at a minute level using weighted averaging or other integration techniques.

Consolidated predictions for the user during the wear period are generated based on the integrated predictions from the combination scheme (block 1310). The consolidated predictions 632, for instance, represent a unified assessment that leverages strengths of both accelerometer and ECG data sources that may provide enhanced accuracy relative to single-modality approaches. In various examples, the consolidated predictions 632 include indications of sleep periods, active periods, and inactive periods throughout the wear period.

The consolidated sleep and activity classifications are then output (block 1312). For example, the analysis module 304 causes display of the consolidated predictions 632 via the presentation module 306 as part of a report 328. In some implementations, the consolidated predictions 632 are incorporated into the report 328 to overlay heart rate data with sleep and activity information. In various examples, the output includes summary statistics for the full wear period and/or daily breakdowns that describe temporal correlations between physiological data and behavioral states.

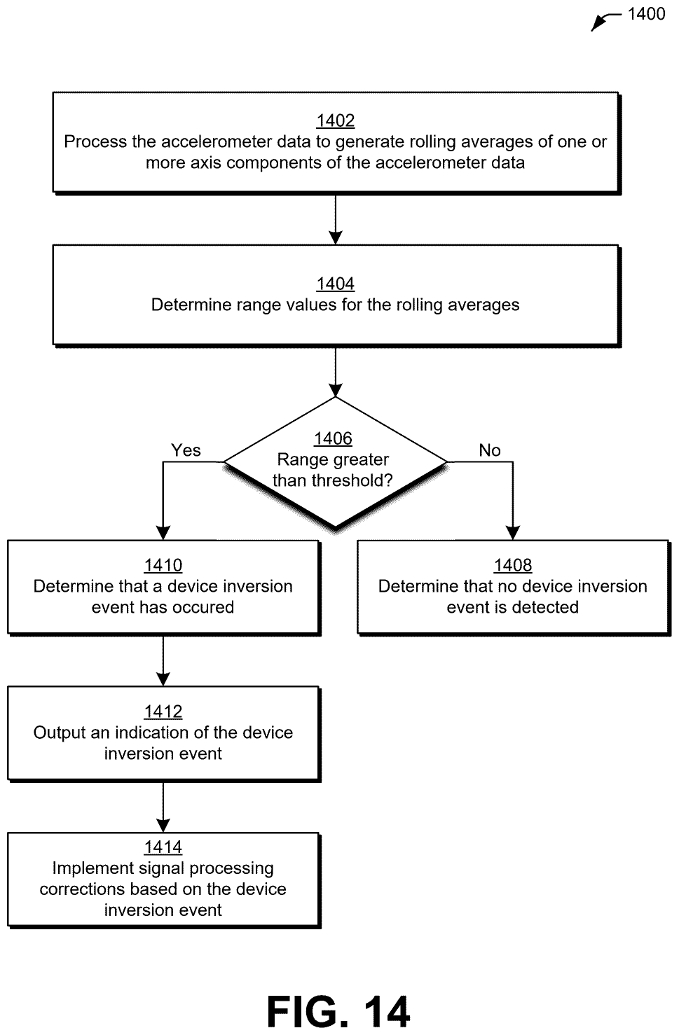

FIG. 14 depicts a flow diagram depicting an algorithm as a step-by-step procedure 1400 in an example implementation that is performable by a processing device to detect device inversion events during extended monitoring periods. The procedure 1400, for instance, can be implemented as one or more substeps of one or more of the preceding flow diagrams.

To begin in this example, accelerometer data is processed to generate rolling averages of axis components (block 1402). In various examples, the procedure 1400 calculates a first rolling average of an x-component of the accelerometer data 310 and a second rolling average of a y-component of the accelerometer data 310 over a predetermined temporal interval, such as a 24-hour period. The rolling averages, for instance, smooth variations associated with typical user activity patterns while preserving long-term trends that indicate device orientation changes.

Range values are then determined for the calculated rolling averages (block 1404). For instance, the prediction system 114 computes a first range of the first rolling average and a second range of the second rolling average. In some cases, the range calculation involves determining a difference between maximum and minimum values of each rolling average component during the wear period. The range values provide quantitative measures of variation in the accelerometer axis components that can indicate whether the device has undergone significant orientation changes during monitoring.

The procedure 1400 then evaluates whether the calculated ranges exceed a predetermined threshold (block 1406). The threshold value, for example, may be set to distinguish between normal positional variations and inversion events. In various implementations, the threshold comparison may involve evaluation of whether either the first range or the second range exceeds the threshold value. In at least one example, the threshold is approximately 0.8.

When the range values do not exceed the threshold (e.g., “No” at block 1406), the procedure 1400 determines that no device inversion event is detected (block 1408). In such cases, the accelerometer data 310 may be processed using standard analysis algorithms without corrections for orientation.

Conversely, when the range values exceed the threshold (e.g., “Yes” at block 1406), the procedure 1400 determines that a device inversion event has occurred (block 1410). The detection of an inversion event may trigger additional analysis to characterize timing and nature of the orientation change. In some embodiments, a number of inversion events may be calculated and particular temporal instances when orientation changes occurred can be identified by analyzing crossing points of the rolling averages relative to calculated flip thresholds.

An indication of the device inversion event is then output (block 1412). For instance, the prediction system 114 generates an indication in substantially real time such as to alert the person 102 that the monitoring device 104 is inverted. Additionally or alternatively, the prediction system 114 causes the device inversion event to be configured for output in a user interface, such as in a report 328.

Further, signal processing corrections are implemented based on the detected device inversion events (block 1414). The corrections, for example, may include automatic adjustment of how the accelerometer data 310 and/or the ECG data 314 is processed. By way of example, processing of ECG signal polarity is modified responsive to detection of an inversion events, as device orientation changes can affect electrical signal characteristics captured by the ECG sensor 312. The signal processing corrections help maintain accuracy of derived insights 116 throughout the wear period despite orientation changes that may occur.

### Machine Learning and AI in Physiological Monitoring Using Low Sample Rate Accelerometer Data

The previous examples describe various instances of artificial intelligence (“AI”) models and/or machine-learning models such as with respect to the machine learning system 326, the trained ASP model 608, and/or the trained ESP model 614. In one or more examples, an AI model, e.g., a machine-learning model, refers to a computer representation that is tunable (e.g., through training and retraining) based on inputs without being actively programmed by a user to approximate unknown functions, automatically and without user intervention. For instance, the term machine-learning model includes a model that utilizes algorithms to learn from, and make predictions on, known data by analyzing training data to learn and relearn to generate outputs that reflect patterns and attributes of the training data.

In the context of physiological monitoring using low sample rate accelerometer data, machine-learning models are implementable (e.g., by one or more processing devices of the prediction system 114) to analyze motion patterns and physiological data to generate insights 116 related to user states and device conditions during extended wear periods. For example, the state module 318, position module 320, orientation module 322, and/or multiparameter module 324 may each utilize one or more machine-learning models to process low sample rate accelerometer data 310 and ECG data 314 collected by the monitoring device 104. Examples of machine-learning models applicable to low sample rate accelerometer analysis include neural networks, convolutional neural networks (CNNs), long short-term memory (LSTM) neural networks, generative adversarial networks (GANs), decision trees (e.g., for sleep/activity state classification), support vector machines, linear regression, logistic regression, Bayesian networks, random forest learning, dimensionality reduction algorithms, boosting algorithms, deep learning neural networks, and so forth.

A machine-learning model, for instance, is configurable using a plurality of layers having, respectively, a plurality of nodes. The plurality of layers are configurable to include an input layer, an output layer, and one or more hidden layers. In the context of low sample rate accelerometer monitoring, the input layer may receive various motion-derived parameters 316 generate based on the accelerometer data 310, such as acceleration magnitude 406, activity parameters 408, reference vectors 508, position vectors 510, rolling averages of accelerometer axis components, and so forth. The hidden layers, for instance, process these inputs through weighted connections to identify complex patterns indicative of user states such as sleep/activity states 118, body positions 120, and device orientations 122, e.g., patterns that are not detectable using conventional high-frequency accelerometer analysis modalities. The output layer may produce classifications indicating sleep states, active states, and inactive states during processing by the state module 318, and/or provide body angle calculations and position classifications. Calculations are performed by the nodes within the layers via hidden states through a system of weighted connections that are “learned” during training of the machine-learning model to implement a variety of physiological assessment tasks under constrained power conditions.

In order to train the machine-learning model for low sample rate accelerometer monitoring, training data is received that provides examples of “what is to be learned” by the machine-learning model, i.e., as a basis to learn patterns from the data. For low sample rate accelerometer applications, the training data may include labeled datasets such as the ASPM training data 604 that includes training accelerometer data 616 with corresponding ground truth labels 618 from users with known sleep and activity states, and the ESPM training data 610 that includes PSG labeled ECG training data 620 and ASPM labeled ECG training data 622. The machine learning system 326 that includes the machine learning model, for instance, collects and preprocesses the training data that includes input features (e.g., motion-derived parameters 316 extracted from 1.56 Hz accelerometer measurements, body angle calculations, device orientation indicators) and corresponding target labels (e.g., “sleep state,” “active state,” “inactive state,” or specific body position classifications such as upright, reclining, or supine orientations).

The machine-learning system 326 is further operable to initialize various parameters of the machine-learning model, which are usable by the machine-learning model as internal variables to represent and process information during training. These parameters are further usable to represent inferences gained through training on low sample rate data patterns that differ significantly from conventional high-frequency accelerometer analysis. In one or more implementations, the training data is separated into batches to improve processing and optimization efficiency of the parameters of the machine-learning model during training, which is particularly beneficial for model accuracy when processing extended time-series data collected over wear periods of one to fourteen days.

The training data is then received as an input by the machine-learning model and used as a basis for generating predictions based on a current state of parameters of layers and corresponding nodes of the model, a result of which is output as output data, e.g., accelerometer-based predictions 626, ECG-based predictions 628, consolidated predictions 632, etc. For example, the prediction system 114 includes machine-learning models such as the trained ASP model 608 and trained ESP model 614 that are trained to recognize patterns in low sample rate accelerometer data 310 and ECG data 314 that correlate with particular user states, which enables the prediction system 114 to generate accurate insights 116.

Training of the machine-learning model can include calculation of a loss function to quantify a loss associated with operations performed by nodes of the machine learning model. The loss function is configurable in various ways to control operation and/or functionality of the machine learning model. For instance, the loss function may be designed to prioritize accuracy in detection of sleep states while minimizing false positives that could lead to incorrect activity classifications during periods of minimal movement. Calculation of the loss function, for instance, includes comparing a difference between predictions specified in the output data (e.g., predicted sleep/activity states 118 or body position classifications) with target labels specified by the training data (e.g., clinically validated sleep states or verified body positions). The loss function is configurable in a variety of ways, examples of which include regret, Quadratic loss function as part of a least squares technique, cross-entropy loss, custom loss functions that incorporate temporal consistency requirements for extended wear period analysis, and so forth.

Configuration of the training data is usable to support a variety of usage scenarios in low sample rate accelerometer monitoring. For example, the machine learning models can be trained to detect specific patterns in motion-derived parameters 316 that indicate various sleep stages, identify movement patterns associated with different activity levels equivalent to walking speeds of 2 mph or greater, recognize device orientation changes that indicate inversion events during the wear period, or detect subtle changes in body positioning that may correlate with sleep quality or health conditions. The models such as the trained ASP model 608 can be configured to operate within computational constraints of wearable devices while providing accurate state classifications throughout extended wear periods. The models can further be integrated through the combination scheme 630 with ECG-based models such as the trained ESP model 614 to provide multimodal insights that leverage strengths of both accelerometer and cardiac monitoring. This adaptive approach enables efficient use of computational resources devoted to machine learning processes while ensuring comprehensive physiological analysis using low sample rate data collection techniques that support extended monitoring without device recharging.

It should be understood that many variations are possible based on the disclosure herein. Although features and elements are described above in particular combinations, each feature or element is usable alone without the other features and elements or in various combinations with or without other features and elements.

What is claimed is:

1. A method comprising:
receiving low sample rate accelerometer data having a sample rate that is below a sample rate threshold collected by a wearable device attached to a skin surface of a chest region of a user during a wear period;
processing the low sample rate accelerometer data to extract one or more motion-derived parameters that characterize temporal movement patterns of the user during the wear period; and
generating, based on the one or more motion-derived parameters, an insight for presentation related to a user state during the wear period.

2. The method of claim 1, wherein the low sample rate accelerometer data is collected at a sample rate of approximately 1.56 Hz and the wear period is between one and fourteen days.

3. The method of claim 1, wherein the insight includes predictions of sleep states, active states, and inactive states of the user during one or more temporal intervals of the wear period.

4. The method of claim 3, wherein the one or more motion-derived parameters include an acceleration magnitude for a particular temporal interval of the wear period calculated as a square root of a sum of squares of one or more acceleration components of the low sample rate accelerometer data and an activity parameter calculated as a standard deviation of the acceleration magnitude for the particular temporal interval, wherein a relatively low standard deviation corresponds to user stillness and a relatively high standard deviation corresponds to user movement.

5. The method of claim 1, wherein the insight includes a body angle or body position of the user during one or more temporal intervals of the wear period.

6. The method of claim 5, wherein the one or more motion-derived parameters include a reference vector that corresponds to an upright position of the user generated based on portions of the low sample rate accelerometer data that indicate relatively high activity, and the body angle is calculated as a polar angle in a spherical coordinate system between the reference vector and a position vector for a particular temporal instance of the wear period.

7. The method of claim 1, wherein the insight includes a detection of inversion events of the wearable device during the wear period and is generated based on motion-derived parameters that include rolling averages of accelerometer axis components of the low sample rate accelerometer data over a particular temporal interval of the wear period.

8. The method of claim 1, further comprising configuring the insight for presentation in a user interface as part of a report that includes:
a summary section that depicts aggregate user state data for the wear period; and
a daily breakdown section that depicts the user state in temporal correlation with physiological data collected during the wear period.

9. The method of claim 1, further comprising receiving electrocardiogram (“ECG”) data collected by the wearable device, and wherein generating the insight is further based on the ECG data.

10. A processing device comprising:
one or more processors; and
memory having stored computer-readable instructions that are executable by the one or more processors to perform operations comprising:
receiving accelerometer data having a sample rate that is below a sample rate threshold, the accelerometer data collected by an accelerometer of a wearable device attached to a skin surface of a user during a wear period;
processing the accelerometer data to extract one or more motion-derived parameters that characterize temporal movement patterns of the user during the wear period; and
generating, based on the one or more motion-derived parameters, an insight for presentation related to a condition of the wear period.

11. The processing device of claim 10, wherein the accelerometer data is collected at a sample rate of approximately 1.56 Hz and the wear period is between one and fourteen days.

12. The processing device of claim 10, wherein the insight includes sleep states, active states, and inactive states of the user throughout the wear period.

13. The processing device of claim 10, wherein the insight includes a body angle or body position of the user throughout the wear period.

14. The processing device of claim 10, wherein the insight includes a detection of whether an inversion event to the wearable device has occurred during the wear period.

15. The processing device of claim 10, the operations further comprising receiving electrocardiogram (“ECG”) data collected by an ECG sensor of the wearable device and generating the insight based in part on the ECG data.

16. The processing device of claim 15, the operations further comprising configuring the insight for presentation in a user interface as part of a report that includes a summary section depicting aggregate user state data for the wear period and a daily breakdown section that includes heart rate data overlaid on sleep and activity data.

17. A system comprising:
an accelerometer sensor of a wearable device configured to collect low sample rate accelerometer data via contact with a skin surface of a user during a wear period; and
one or more processors configured to:
receive the low sample rate accelerometer data, the low sample rate accelerometer data having a sample rate that is below a sample rate threshold;
process the low sample rate accelerometer data to extract one or more motion-derived parameters that characterize temporal movement patterns of the user during the wear period; and
present an insight generated based on the one or more motion-derived parameters that indicates a user state during the wear period.

18. The system as described in claim 17, wherein the insight includes one or more of a sleep or activity state of the user, a body position of the user, or a device inversion event of the wearable device during the wear period.

19. The system as described in claim 17, further comprising one or more electrocardiogram (ECG) sensors of the wearable device, wherein the one or more processors are configured to receive ECG data collected by the ECG sensor and determine the insight based on the ECG data and the low sample rate accelerometer data.

20. The system as described in claim 17, wherein the one or more processors are configured to process the low sample rate accelerometer data by applying a trained machine learning algorithm that has been trained on historical accelerometer data and corresponding user state labels to extract the one or more motion-derived parameters and generate the insight.
